(19) United States
(12) Patent Application Publication
(10) Pub. No.: US 2011/0152252 A1
(43) Pub. Date: Jun. 23, 2011

(54) SHIGA TOXIN B-SUBUNIT/CHEMOTHERAPEUTICS CONJUGATES

(75) Inventors: Ludger Johannes, Courbevoie (FR); Abdessamed El Alaoui, Montreuil (FR); Didier Decaudin, Verriere Le Buisson (FR); Sylvie Robine, Vanves (FR); Frederic Schmidt, Vincennes (FR); Jean-Claude Florent, Gif Sur Yvette (FR)

(73) Assignees: INSTITUT CURIE, PARIS (FR); CENTRE NATIONAL DE LA RECHERCHE SCIENTIFIQUE (CNRS), PARIS (FR)

(21) Appl. No.: 11/958,576

(22) Filed: Dec. 18, 2007

(51) Int. Cl. A61K 31/5513; C07D 491/14; C07D 243/14; A61K 31/437; A61P 35/00

(52) U.S. Cl. 514/221; 546/48; 540/504; 514/283

(57) ABSTRACT

The present invention relates to the use of a Shiga toxin B-subunit moiety as carrier for therapeutic agents, for example, anti-cancer agents such as anti-cancer agents that require intracellular uptake to exert their anti-cancer effects. In particular, the present invention provides conjugates comprising a Shiga toxin moiety covalently linked to an anti-cancer agent through a self-immolative spacer, and methods of using such conjugates to increase cellular uptake and/or specificity for cancer cells of the anti-cancer drug. Also provided are methods of treatment involving administration of such conjugates, and pharmaceutical compositions and kits useful for carrying out such methods of treatment.

### BACKGROUND OF THE INVENTION

The clinical use of chemotherapeutic agents against malignant tumors is successful in many cases but also has several limitations (B. A. Chabner and T. G. Roberts, Nature Rev. Cancer, 2005, 5: 65-72). In particular, anti-cancer drugs often do not affect tumor cells selectively over healthy cells, which leads to high toxicity and side effects (M. V. Blagosklonny, Trends Pharmacol. Sci., 2005, 26: 77-81). Tissues with high cellular division rates (e.g., bone marrow, intestinal mucosa, and the hair follicle cells) are particularly affected. The lack of selectivity and resulting adverse toxicity limit the dose of drug that can be administered to a patient, and therefore the therapeutic potential of certain anti-cancer drugs.

Lack of selectivity is only one, albeit major, obstacle hindering the optimization of tumor drug effectiveness. The efficiency of chemotherapeutic drugs may also be seriously limited by the presence or development of cellular drug resistance (M. Pomeroy and M. Moriarty, Cytotechnology, 1993, 12: 385-391; G. Giaccone and H. M. Pinedo, The Oncologist, 1996, 1: 82-87; M. M. Gottesman, Ann. Rev. Medicine, 2002, 53: 615-627; G. D. Kruth, Oncogene, 2003, 22: 7262-7264). Resistance to a cytostatic/cytotoxic agent can operate by different mechanisms including reduced intracellular accumulation due to decrease or loss of plasma membrane carriers that results in certain anti-cancer drugs being prevented from entering cells and/or increase in the level of energy-dependent pumps such as p-glycoprotein resulting in extrusion of the drug from tumor cells, premature inactivation of the drug leading to insufficient concentration at the target site, impaired activation of the drug due to decrease in or loss of specific enzymatic activities, formation of inactivating antibodies, and appearance of DNA repair mechanisms.

Another limitation of certain chemotherapeutics is their intrinsic low solubility in water. The membrane permeability and efficacy of such drugs increases with increasing hydrophobicity. In addition, parenteral administration of these hydrophobic agents is associated with some problems. Thus, intravenous administration of aggregates formed by undissolved drug in aqueous media can cause embolization of blood capillaries before the drug penetrates a tumor. Additionally, the low solubility of hydrophobic drugs in combination with excretion and metabolic degradation hinders the maintenance of therapeutically significant systemic concentrations.

Although drug delivery systems have been developed with the goal of optimizing anti-tumor drug effectiveness, these systems (e.g., micelles, liposomes, microparticles, antibodies and drug-polymer conjugates) suffer from limitations including instability in the plasma, susceptibility to oxidation or other degradation mechanisms, technical problems with their production, rapid scavenging by reticuloendothelial cells, absence of or low selectivity for cancer cells, and limited cellular internalization.

Therefore, there is still a need in the art for improved drug-delivery approaches to overcome the above-mentioned problems and substantially enhance the efficiency of cancer treatment. Particularly desirable is the development of drug carriers or vehicles that can selectively deliver the drug to the critical target site.

### SUMMARY OF THE INVENTION

The present invention is directed to new systems and strategies for improved delivery and administration of chemotherapeutics. More specifically, the present invention provides methods and compositions for the selective targeting of anti-cancer drugs to intracellular sites. In particular, the present invention encompasses the recognition that the non-toxic B-subunit of Shiga toxin (i) exhibits high specificity for cancer cells expressing the cell surface glycophingolipid receptor, globotriaosyl ceramide, Gb3, (ii) undergoes efficient cellular internalization and is transported in a retrograde fashion from the plasma membrane to the endoplasmic reticulum, via endosomes and the Golgi apparatus, and (iii) can reach Gb3-expressing tumors in vivo. Accordingly, the present invention relates to the use of Shiga toxin B-subunit moieties as selective carriers for chemotherapeutic agents.

More specifically, in one aspect, the present invention provides a conjugate comprising at least one Shiga toxin B-subunit moiety, or a functional equivalent thereof, that is covalently attached to at least one chemotherapeutic moiety through a linker, wherein the linker comprises a self-immolative spacer. In such conjugates, the Shiga toxin B-subunit moiety, which preferentially interacts with cancer cells that over-express the receptor Gb3, plays the role of targeting moiety and delivers the chemotherapeutic moiety to intracellular sites, such as membranes of the biosynthetic/secretory pathway.

In light of the high Gb3 expression levels in tumors—more than 10⁷ binding sites per cancer cells (T. Falguières et al., Mol. Biol. Cell., 2001, 12: 2453)—and preferential retrograde transport in tumor cells when compared to non-tumoral Gb3-expressing cells (T. Falguières et al., Mol. Biol. Cell., 2001, 12: 2453; M. Warnier et al., Kidney Int., 2006, 70: 2085), conjugates of the present invention constitute a new approach for improved selectivity of cancer chemotherapy.

Preferably, a self-immolative spacer within an inventive conjugate is selected for its ability to undergo selective chemical or enzymatic cleavage within target cells. In certain embodiments, the self-immolative spacer undergoes selective enzymatic cleavage. In other embodiments, the self-immolative spacer is selectively cleaved in reductive conditions. For example, the self-immolative spacer may be selectively cleaved in the presence of glutathione, an antioxidant that exhibits higher intracellular concentrations than plasma concentrations and that is present in larger amounts in cancer cells than in normal cells.

Thus, conjugates of the present invention act as prodrugs, i.e., compounds that are converted to drugs (active therapeutic compounds) in vivo by certain chemical or enzymatic modifications of their structure. For purposes of reducing toxicity and side effects and enhancing efficacy, this conversion is preferably confined to the intracellular site of action or target tissue rather than the circulatory system or non-target tissue.

In addition, by varying the nature (chemical structure) of the self-immolative spacer, one may design conjugates that exhibit different stability properties in vivo. For example, a self-immolative spacer may be designed that combines stability in serum with efficient release after uptake by tumor cells. Alternatively, a self-immolative spacer may be designed that combines stability in serum with slow release after uptake by tumor cells. Such a spacer may be advantageously used to prolong the effect of a drug as slow release will sustain the continued presence of the chemotherapeutics in dividing cancer cells while the conjugate is rapidly cleared from the circulation.

Thus, hallmarks of the tumor targeting approach provided by the present invention include: capacity to cross tissue barriers, escape from extracellular inactivation, high numbers of Gb3 receptors on tumor cells, escape from intracellular degradation through retrograde transport, and stable association with cancer cells leading to efficient conversion of prodrug into drug.

There is no limitation on chemotherapeutics that are suitable for use in conjugates of the present invention. Thus, suitable chemotherapeutics include any drugs or agents that can be used in the treatment of cancer or cancer conditions. For example, chemotherapeutics may be selected from the groups consisting of alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones, and anti-androgens. Chemotherapeutics may also be selected among pro-apoptotic agents. For example, pro-apoptotic agents may be ligands to mitochondrial peripheral benzodiazepine receptor (mPBR). For therapeutic intervention, it is needed to find specific ligands that do not interfere with the benzodiazepine receptors of the central nervous system.

Chemotherapeutics suitable for use in the present invention may have any cytotoxic activity when administered alone (i.e., in an unconjugated form). For example, the cytotoxic activity of a chemotherapeutic moiety may be between about 1 nM and 100 μM. In certain embodiments, chemotherapeutics are selected among anti-cancer drugs or agents that exhibit relatively low activity (in the upper nM range). This distinguishes the targeting approach provided by the present invention from the antibody-based targeting technology for which there is a consensus in the field that antibodies need to be used in combination with highly cytotoxic compounds.

In another aspect, the present invention provides pharmaceutical compositions. More specifically, a pharmaceutical composition according to the present invention comprises an effective amount of at least one conjugate provided herein and at least one pharmaceutically acceptable carrier or excipient. Pharmaceutical compositions may be formulated for any route of administration. In certain embodiments, the pharmaceutical composition is formulated to be administered intravenously or orally.

In certain embodiments, a pharmaceutical composition further comprises an additional therapeutic agent. For example, the therapeutic agent may be selected from the group consisting of an analgesic, an anesthetic, a haemostatic agent, an antimicrobial agent, an antibacterial agent, an antiviral agent, an antifungal agent, an antibiotic, an anti-inflammatory agent, an antioxidant, an antiseptic agent, an antihistamine agent, an antipruritic agent, an antipyretic agent, an immunostimulating agent, a dermatological agent, an anti-cancer agent, and any combination thereof.

In another aspect, the present invention provides methods of treating cancer or a cancerous condition in a subject (e.g., human or another mammal). A method according to the present invention generally comprises a step of: administering to the subject an effective amount of a conjugate described herein. Administration may be carried out using any route of administration. In certain embodiments, administration is carried out by intravenous or oral administration. Methods of the present invention may be used to treat any of a wide variety of cancers or cancerous conditions. In certain embodiments, methods of treatment of the present invention are used to treat cancers and cancerous conditions associated with overexpression of the receptor Gb3. Examples of such cancers and cancerous conditions include, but are not limited to, lymphomas, ovarian cancers, breast tumors, testicular cancers, colorectal cancers, intestine tumors, and astrocytomas.

In certain embodiments, methods of treatment of the present invention further comprise a step of: administering to the subject an additional therapeutic agent. For example, the therapeutic agent may be selected from the group consisting of an analgesic, an anesthetic, a haemostatic agent, an antimicrobial agent, an antibacterial agent, an antiviral agent, an antifungal agent, an antibiotic, an anti-inflammatory agent, an antioxidant, an antiseptic agent, an antihistamine agent, an antipruritic agent, an antipyretic agent, an immunostimulating agent, a dermatological agent, an anti-cancer agent, and any combination thereof. The therapeutic agent may be administrated prior to, concomitantly with, and/or following administration of the conjugate.

In yet another aspect, the present invention provides a method for increasing the selectivity of a chemotherapeutic agent, the method comprising a step of: covalently attaching the chemotherapeutic agent to a Shiga toxin B-subunit moiety, or a functional equivalent thereof, through a linker to form a conjugate, wherein the linker comprises a self-immolative spacer as described herein. Formation of a conjugate according to the present invention may result in increased specificity of the chemotherapeutic agent for cancer cells (for example, cancer cells that express Gb3), and/or increased cellular uptake by cancer cells (for example via a retrograde pathway).

These and other objects, advantages and features of the present invention will become apparent to those of ordinary skill in the art having read the following detailed description of the preferred embodiments.

### BRIEF DESCRIPTION OF THE DRAWING

FIG. 1 shows examples of cleavage reactions of Shiga toxin B-subunit (STxB) conjugates leading to release of a drug in the case of (A) an aromatic self-immolative spacer and (B) an aliphatic self-immolative spacer.

FIG. 2 is a scheme presenting the principle of retrograde delivery applied to a STxB/SN-38 conjugate according to the present invention.

FIG. 3 is a scheme presenting cleavage reactions of compounds 2 (more specifically, compounds 2a and 2b) and 3 (more specifically, compounds 3a and 3b). These reactions lead to release of the chemotherapeutics SN-38 (compound 1) by compounds 2a and 3a and of a biotin derivative (compound 4) by compounds 2b and 3b. The double-headed arrows indicate the bonds that are cleaved first.

FIG. 4 is a scheme presenting the synthesis of compounds 3a and 3b, as described in Example 1.

FIG. 5 is a graph presenting the results of an ELISA analysis of the activation of compound 3b in HT-29 cells. HT-29 cells were incubated in the presence of compound 3b (1 μM) on ice. After washing, the cells were shifted to 37° C. for the indicated times, lysed, and the lysates were analyzed by ELISA for the indicated markers. Means of two determinations are shown. t indicates the incubation time.

FIG. 6 is a set of fluorescence microscopy pictures showing the activation of compound 3b after retrograde trafficking to the biosynthetic/secretory pathway. HT-29 cells were incubated in the presence of compound 3b (1 μM) on ice. After washing, the cells were shifted to 37° C. for the indicated times, fixed, and stained for the indicated markers. Top row: 45 minute-uptake; Bottom row: 48 hour-uptake; Biotin: green; STxB: red; Rab6 (Golgi): blue.

FIG. 7 is a graph showing the cytotoxic activity of different compounds in HT-29 cells. The indicated compounds were incubated for 6 hours at 37° C. with HT-29 (with or without PPMP) or CHO cells. After washing, incubation was continued for 7 days at 37° C., followed by live-cell counting by using 3-(4,5-dimethylthiazole-2-yl)-2,5-diphenyltetrazolium bromide (MTT).

FIG. 8 shows results of the pathological analysis of tumor lesions and stromal reaction in STxB-SN38 injected APC^{1638N} mice (see Example 3). Strong signs of inflammation were observed in animals in which tumors had completely disappeared (left), or in which a residual adenoma could be detected (middle). In contrast, the inflammatory response was weak in an animal which apparently did not respond to the treatment (right) and that still showed the presence of a high grade adenoma.

FIG. 9 show results of the pathological analysis of tumor lesions and stromal reaction in CPT11 injected APC^{1638N} mice (see Example 3). A very moderate inflammatory reaction was observed, except for the case of carcinoma in which a strong infiltration from the stroma was observed.

### DEFINITIONS

For purpose of convenience, definitions of a variety of terms used throughout the specification are presented below.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is the full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains, such as oxidation of sulfhydryl groups. Thus, the term “protein” (or its equivalent terms) is intended to include the amino acid sequence of the full-length native protein, subject to those modifications that do not change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative slicing or limited proteolysis), or in the alternative, may arise from differential post-translational modification (e.g., glycosylation, acylation or phosphorylation).

The term “protein analog”, as used herein, refers to a polypeptide that possesses a similar or identical function as a parent polypeptide but need not necessarily comprise an amino acid sequence that is similar or identical to the amino acid sequence of the polypeptide, or possess a structure that is similar or identical to that of the polypeptide. Preferably, in the context of the present invention, a protein analog has an amino acid sequence that is at least about 30%, more preferably, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 99%) identical to the amino acid sequence of the parent polypeptide, and/or contains a characteristic sequence of the parent. Moreover, those of ordinary skill in the art will understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than about 90%, 96%, 97%, 98% or 99% in one or more highly conserved regions usually encompassing at least 3-4 and often up to 20 or more amino acids, with the parent polypeptide, is encompassed in the term “protein analog”.

The term “protein fragment”, as used herein, refers to a polypeptide comprising an amino acid sequence of at least 5 amino acid residues of the amino acid sequence of a second polypeptide. The fragment of a protein may or may not possess a functional activity of the full-length native protein.

The term “biologically active”, when used herein to characterize a protein variant, analog or fragment, refers to a molecule that shares sufficient amino acid sequence identity with the protein to exhibit similar or identical properties than the protein (e.g., ability to specifically bind to the glycolipid Gb3 and/or to be internalized into cells expressing Gb3).

The term “homologous” (or “homology”), as used herein, refers to a degree of identity between two polypeptides, molecules or between two nucleic acid molecules. When a position in both compared sequences is occupied by the same base or amino acid monomer subunit, then the respective molecules are homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid residues. Similar residues are conservative substitutions for, or “allowed point mutations” of, corresponding amino acid residues in a reference sequence. “Conservative substitutions” of a residue in a reference sequence are substitutions that are physically or functionally similar to the corresponding reference residue, e.g., that have a similar size, shape, electric charge, chemical properties, including the ability to form covalent or hydrogen bonds, or the like. Particularly preferred conservative substitutions are those fulfilling the criteria defined for an “accepted point mutation” by Dayhoff et al. (“Atlas of Protein Sequence and Structure”, 1978, Nat. Biomed. Res. Foundation, Washington, D.C., Suppl. 3, 22: 354-352).

The term “isolated”, when used herein in reference to a protein or polypeptide, means a protein or polypeptide, which by virtue of its origin or manipulation is separated from at least some of the components with which it is naturally associated or with which it is associated when initially obtained. By “isolated”, it is alternatively or additionally meant that the protein or polypeptide of interest is produced or synthesized by the hand of man.

The terms “self-immolative spacer” and “auto-destructive spacer” are used herein interchangeably. They refer to a chemical moiety which is bound through two bonds to two molecules and which eliminates itself from the second molecule if the bond to the first molecule is cleaved.

The term “aliphatic” or “aliphatic group”, as used herein, denotes a hydrocarbon moiety that may be straight-chain (i.e., unbranched), branched or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In certain embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments, aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl).

The terms “individual” and “subject” are used herein interchangeably. They refer to a human or another mammal (e.g., mouse, rat, rabbit, dog, cat, cattle, swine, sheep, horse or primate) that can be afflicted with or is susceptible to cancer or a cancerous condition but may or may not have the disease or condition. In many embodiments, the subject is a human being. The terms “individual” and “subject” do not denote a particular age, and thus encompass adults, children, and newborns.

As used herein, the term “cancer cell” refers to a cell in a mammal (e.g., a human being) in vivo which undergoes undesired and unregulated cell growth or abnormal persistence of abnormal invasion of tissues. In vitro this term also refers to a cell line that is a permanently immortalized established cell culture that will proliferate indefinitely and in an unregulated manner, if given appropriate fresh medium and space. In certain embodiments of the present invention, cancer cells express Gb3.

The terms “normal” and “healthy” are used herein interchangeably. When used in connection with an individual or group of individuals, they refer to an individual or group of individuals who do not have cancer or a cancer condition. When used in connection with cells or biological tissues, they refer to non-cancerous cells or non-cancerous tissues.

As used herein, the terms “cancer” or “cancerous condition” refer to or describe a physiological condition in mammals that is typically characterized by unregulated cell growth. Examples of cancers include, but are not limited to, carcinoma, lymphoma, blastoma, sarcoma, and leukemia. More particularly, examples of such cancers include lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma.

The term “treatment” is used herein to characterize a method or process that is aimed at (1) delaying or preventing the onset of a disease or condition (e.g., cancer); (2) slowing down or stopping the progression, aggravation, or deterioration of the symptoms of the disease or condition; (3) bringing about ameliorations of the symptoms of the disease or condition; or (4) curing the disease or condition. A treatment may be administered prior to the onset of the disease, for a prophylactic or preventive action. Alternatively or additionally, a treatment may be administered after initiation of the disease or condition, for a therapeutic action.

A “pharmaceutical composition” is defined herein as comprising an effective amount of at least one agent of the invention (i.e., a toxin-chemotherapeutics conjugate) and at least one pharmaceutically acceptable carrier.

As used herein, the term “effective amount” refers to any amount of a compound, agent or composition that is sufficient to fulfil its intended purpose(s), e.g., a desired biological or medicinal response in a tissue, system or subject. For example, in certain embodiments of the present invention, the purpose(s) may be: to specifically deliver a drug to a target tissue, to deliver a drug inside a cell (e.g., a cancer cell), to slow down or stop the progression, aggravation, or deterioration of the symptoms of a disease (e.g., cancer), to bring about amelioration of the symptoms of the disease and/or cure the disease.

The term “pharmaceutically acceptable carrier or excipient” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, adsorption delaying agents, and the like. The use of such media and agents for pharmaceutically active substances is well known in the art (see for example, “Remington's Pharmaceutical Sciences”, E. W. Martin, 18^{th} Ed., 1990, Mack Publishing Co.: Easton, Pa., which is incorporated herein by reference in its entirety).

The terms “therapeutic agent” and “drug” are used herein interchangeably. They refer to a substance, molecule, compound, agent, factor or composition effective in the treatment of a disease or condition.

The term “cytotoxic”, when used herein to characterize a moiety, a compound, a drug or an agent refers to a moiety, a compound, a drug or an agent that inhibits or prevents the function of cells and/or causes destruction of cells.

The terms “chemotherapeutics” and “anti-cancer agents or drugs” are used herein interchangeably. They refer to those medications that are used to treat cancer or cancerous conditions. Anti-cancer drugs are conventionally classified in one of the following groups: alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. Anti-cancer drugs are generally given in a particular regimen over a period of weeks. Certain chemotherapeutic medications have the ability to directly kill cancer cells.

The term “small molecule” includes any chemical or other moiety that can act to affect biological processes. Small molecules can include any number of therapeutic agents presently known and used, or can be small molecules synthesized in a library of such molecules for the purpose of screening for biological function(s). Small molecules are distinguished from macromolecules by size. Small molecules suitable for use in the present invention usually have molecular weight less than about 5,000 daltons (Da), preferably less than about 2,500 Da, more preferably less than about 1,000 Da, most preferably less than about 500 Da. In some embodiments, small molecules are not polymers.

The terms “approximately” and “about”, as used herein in reference to a number, generally includes numbers that fall within a range of 10% in either direction of the number (greater than or less than the number) unless otherwise stated or otherwise evident from the context (except where such a number would exceed 100% of a possible value).

As used herein, the term “physiologically tolerable salt” refers to any acid addition or base addition salt that retains the biological activity and properties of the corresponding free base or free acid, respectively, and that is not biologically or otherwise undesirable. Acid addition salts are formed with inorganic acids (e.g., hydrochloric, hydrobromic, sulfuric, nitric, phosphoric acids, and the like); and organic acids (e.g., acetic, propionic, pyruvic, maleic, malonic, succinic, fumaric, tartaric, citric, benzoic, mandelic, methanesulfonic, ethanesulfonic, p-toluenesulfonic, salicylic acids, and the like. Base addition salts can be formed with inorganic bases (e.g., sodium, potassium, lithium, ammonium, calcium, magnesium, zinc, aluminium salts, and the like) and organic bases (e.g., salts of primary, secondary and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethyl-aminoethanol, 2-diethylaminoethanol, trimethamine, dicyclohexyl-amine, lysine, arginine, histidine, caffeine, procaine, hydrabanine, choline, betaine, ethylene-diamine, glycosamine, methylglucamine, theobromine, purines, piperazine, N-ethylpiperidine, polyamine resins, and the like).

### DETAILED DESCRIPTION OF CERTAIN PREFERRED EMBODIMENTS

As mentioned above, the present invention provides compositions and methods for increasing the efficacy of drugs by improving their delivery to the target site. In particular, the present invention provides toxin-chemotherapeutics conjugates, and methods for using these conjugates in the treatment of cancer.

### I—Toxin-Chemotherapeutics Conjugates

Conjugates according to the present invention generally comprise at least one Shiga toxin B-subunit moiety, or a functional equivalent thereof, covalently attached to at least one chemotherapeutic moiety through a linker, wherein the linker comprises a self-immolative spacer.

### A. Self-Immolative Spacers

Within a conjugate of the present invention, a self-immolative spacer moiety spaces and covalently links together a toxin moiety and a chemotherapeutic moiety. A self-immolative spacer may be defined as a bifunctional chemical moiety which is capable of covalently linking together two spaced chemical moieties into a normally stable tripartite molecule, releasing one of said chemical moieties from the tripartite molecule by means of cleavage; and following said cleavage, spontaneously cleaving the remainder of the molecule to release the other of said spaced chemical moieties.

In certain preferred embodiments of the present invention, the self-immolative spacer is chosen for its ability to be selectively cleaved at the target site or target cell, i.e., at or in the vicinity of the site of therapeutic action or activity of the chemotherapeutic agent. The cleavage may be enzymatic or chemical (e.g., reductive or pH conditions) in nature. The term “selective”, as used herein in connection with the enzymatic or chemical cleavage, means that the rate of cleavage of the spacer by the enzyme or chemical conditions is greater than its rate of cleavage by other enzymes or chemical conditions. This feature allows the chemotherapeutic agent that is relatively innocuous to cells while still in the conjugated form to be transported through the system without decomposition of the conjugate and to be delivered at the target site where, in the presence of the “cleaving” enzyme or conditions, it is selectively released to its pharmacologically active form. In this regard, a conjugate of the present invention acts as a prodrug. This aids in reducing systemic activation of the chemotherapeutic agent, reducing toxicity, reducing side effects, and enhancing the efficacy of the chemotherapeutic agent by increasing its concentration at the target site.

Exemplary mechanisms by which cleavage of a spacer may release a chemotherapeutic moiety from the toxin moiety in an inventive conjugate include hydrolysis in the acidic pH of lysosomes (hydrazones, acetals, and cis-aconitate-like amides), peptide cleavage by lysosomal enzymes (e.g., the capthepsins and other lysosomal enzymes), and reduction of disulfides (e.g., by glutathione). Self-immolative spacers whose cleavage is based on these different mechanisms have been designed and are known in the art. Examples of such self-immolative spacers include, but are not limited to, those described in U.S. Pat. Nos. 5,773,001; 5,739,116; 5,877,296; 5,728,868; 5,770,731; 6,214,345; 6,218,519; 6,268,488; 7,091,186; 7,232,805; and 7,235,585; and PCT Publication No. WO 2005/0112919.

Thus, in certain embodiments, a conjugate of the present invention comprises at least one Shiga toxin B-subunit, or a functional equivalent thereof, covalently attached to chemotherapeutic moiety through a self-immolative spacer, wherein the spacer is hydrolysable in acidic pH. In other embodiments, the spacer is enzymatically cleavable. In yet other embodiments, the spacer is cleavable by reductive conditions. The self-immolative linker may be a substituted or unsubstituted alkyl, substituted or unsubstituted aryl, a substituted or unsubstituted heteroaryl, or a substituted or unsubstituted heteroalkyl group. Selection of a self-immolative spacer for release of a chemotherapeutic agent to or in the vicinity of a cellular target is within the knowledge of one skilled in the art.

In certain preferred embodiments of the present invention, self-immolative spacers comprise a cleavable disulfide bond (—S—S—). In cells, disulfide bonds are cleaved by reductors, generally, glutathione. Glutathione (L-glutamyl-L-cysteinyl-glycine) is a small protein composed of three amino acids: cysteine, glutamic acid, and glycine. Glutathione (GSH) is found almost exclusively in its reduced form, since the enzyme which reverts it from its oxidized form (GSSG), glutathione reductase, is constitutively active and inducible upon oxidative stress. Glutathione occurs in different species of animals, plants, and prokaryotes, and is naturally present inside almost every cell of the human body. One of the primary biological functions of glutathione is to act as a non-enzymatic reducing agent to help keep cysteine thiol side chains in a reduced state on the surface of proteins. Gluthatione also plays a role in the prevention of oxidative stress in most cells and helps trap free radicals that can damage biomolecules such as DNA and RNA. Glutathione is the main anti-oxidant of the cytosol, with a concentration between 5 and 10 mM and a molar GSH/GSSG ratio of 100 (Go Saito et al., Adv. Drug Deliv. Rev., 2003, 55: 199-215).

In addition to exhibiting higher intracellular concentrations than plasma concentrations, glutathione is found in larger quantities in cancer cells than in normal cells (J. Kigawa et al., Cancer, 1998, 82: 697-702). This has led research groups to focus their efforts on the design and development of spacers comprising disulfide bonds cleavable by glutathione. Examples of such spacers include, but are not limited to, the spacer of Mylotarg™, the first targeted chemotherapeutics (calicheamycin) that has received FDA approval (A. L. Smith and K. C. Nicolaou, J. Med. Chem., 1996, 39: 2103-2117; P. R. Hamman et al., Bioconjug. Chem., 2002, 13: 40-46; E. L. Sievers and M. Linenberger, Curr. Opin. Oncol., 2001, 55: 522-527; L. L. Himman et al., J. Cancer Res., 1993, 53: 3336-3342), and the spacers comprised in immunoconjugates of ricin A (P. L. Amlot et al., Blood, 1993, 82: 2624-2633; R. Shnell et al., Leukemia, 2000, 14: 129-135), maytansine derivatives (C. Liu et al., Proc. Natl. Acad. Sci. USA, 1996, 93: 8618-8623), and CC-1065 derivatives (R. V. Chari et al., Cancer Res., 1995, 55: 4079-4084).

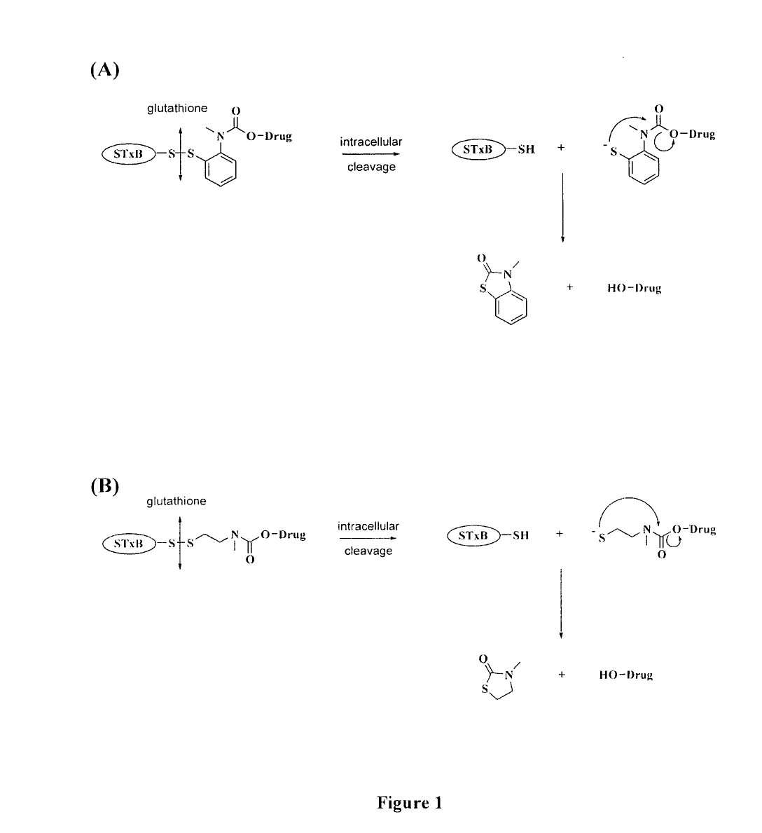

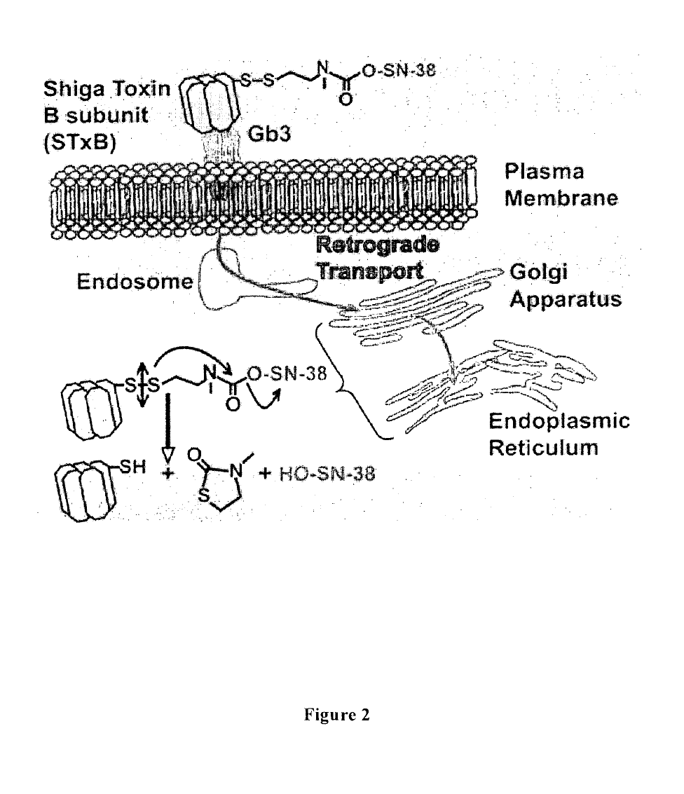

In certain embodiments, the self-immolative spacer comprises a disulfide bond and an aromatic spacer moiety that participates in the self-destruction of the spacer after cleavage of the disulfide bond. In such spacers, self-immolation may proceed, for example, through intramolecular cyclization or via a 1,4- or 1,6 elimination. Examples of such self-immolative spacers include, but are not limited to, those described in T. H. Fife et al., J. Am. Chem. Soc., 1975, 5878-5882; P. D. Senter et al., J. Org. Chem., 1990, 55: 2975-2978; Wakselman, Nouveau Journal de Chimie, 1983, 4: 439). FIG. 1(A) shows a cleavage reaction undergone by an inventive STxB conjugate comprising an aromatic disulfide self-immolative spacer.

In other embodiments, the self-immolative spacer comprises a disulfide bond and an aliphatic spacer moiety. In such spacers, self-immolation may proceed, for example, through intramolecular cyclization. FIG. 1(B) shows a cleavage reaction undergone by an inventive STxB conjugate comprising an aliphatic disulfide self-immolative spacer.

As will be recognized by one skilled in the art, the stability of a conjugate of the present invention may be tuned by selection of the self-immolative spacer. More specifically, conjugates may be designed that exhibit various degrees of stability. For example, self-immolative spacers may be selected to impart to the conjugate a high stability in serum and allow a rapid intracellular release of the chemotherapeutic agent. Alternatively, self-immolative spacers may be selected to impart to the conjugate a high stability in serum and allow a slow release of the chemotherapeutic agent. A slow release of the chemotherapeutic agent should sustain the continued presence of the active principle in dividing cancer cells, with the conjugate being otherwise rapidly cleared from the circulation.

In general, a self-immolative spacer is covalently linked at one of its ends to the Shiga toxin B-subunit moiety (or functional equivalent thereof) and covalently linked at its other end to the chemotherapeutic moiety. Preferably, covalent binding of the spacer to the chemotherapeutic agent inhibits pharmaceutical activity. Covalent binding between the spacer and toxin moiety and between the spacer and chemotherapeutic agent can be achieved by taking advantage of reactive functional groups present on the toxin moiety, chemotherapeutic moiety and/or spacer. Alternatively or additionally, reactive functional groups may be added to the toxin moiety, chemotherapeutic moiety and/or spacer to allow binding. Reactive functional groups may be selected from a wide variety of chemical groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids, isonitriles; amidines, imides, imidates, nitrones, hydroxylamines, oximes, hydroxamic acids, thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those usually used to prepare bioconjugates, e.g., N-hydroxysuccinimide esters, maleimides and the like (see, for example, Hermanson, “Bioconjugate Techniques”, Academic Press: San Diego, 1996). Methods to introduce each of these functional groups are well known in the art and their application to or modification for a particular purpose is within the ability of one of skill in the art (see, for example, Sandler and Karo, Eds., “Organic Functional Group Preparations”, Academic Press: San Diego, 1989). Reactive functional groups may be protected or unprotected.

### B. Toxins

Conjugates of the present invention comprise at least one Shiga toxin B-subunit moiety or a functional equivalent thereof.

Shiga toxin is a bacterial toxin of the AB5 family that is secreted by the disentry-causing bacterium Shigella dysenteriae (K. Sandvig and B. van Deurs, Annu. Rev. Cell Devel. Biol., 2002, 18: 1-24; J. Gariepy, Crit. Rev. Oncol./Hematol., 2001, 39: 99-106; K. Sandvig, Toxicon., 2001, 39: 1629-1635; and D. G. Pina and L. Johannes, Toxicon., 2005, 45: 389-393). It is composed of an enzymatic A-subunit and a non-toxic B-subunit. The A-subunit modifies ribosomal RNA thus leading to inhibition of protein synthesis in higher eukaryotic target cells after transferring into the cytoplasm of these cells. For cellular binding and intracellular transport, the A-subunit has to interact with the B-subunit. The B-subunit is a homopentamer protein that mediates binding to and internalization into target cells by interacting with the cell surface glycolipid receptor Gb3, also called CD77. The B-subunit alone conserves the intracellular transport characteristics of the holotoxin which, in many Gb3-expressing cells, is transported in a retrograde fashion from the plasma membrane to the endoplasmic reticulum, via the early endosome and the Golgi apparatus.

The retrograde trafficking pathway from endosomes to the Golgi apparatus and endoplasmic reticulum is of special importance since it provides a route to deliver drugs, bypassing the acid pH, hydrolytic environment of the lysosome. Furthermore, Gb3 is highly expressed in tumors, with more than 10⁷ binding sites per cancer cells (T. Falguières et al., Mol. Biol. Cell., 2001, 12: 2453).

The present Applicants have shown that the Shiga toxin B-subunit (STxB) can reach Gb3-expressing tumors in vivo (K. P. Janssen et al., Cancer Res., 2006, 14: 7230-7236). More specifically, after oral or intravenous injections in mice, STxB was found to target spontaneous digestive Gb3-expressing adenocarcinomas, while nontumoral mucosa was devoid of labeling with the exception of rare enteroendocrine and CD11b-positive cells. As opposed to other delivery tools that are often degraded or recycled on cancer cells, the B-subunit stably associates with these cells due to its trafficking via the retrograde transport route.

Thus, in conjugates of the present invention, the toxin moiety plays the role of targeting moiety. As a delivery tool, STxB exhibits characteristics that the protein has acquired as an intestinal pathogen in co-evolution with its hosts: stability at extreme pH and in the presence of proteases, capacity to cross tissue barriers and to distribute in the organism, and resistance against extra- and intracellular activation (L. Johannes and D. Decaudin, Gene Ther., 2005, 12: 1360).

In certain embodiments, the targeting moiety comprises the B-subunit of Shiga toxin, which has the polypeptide sequence described in N. A. Stockbine et al., J. Bacteriol., 1988, 170: 1116-1122 (see also International Patent Publication No. WO 09/03881; European Pat. No. EP 1 386 927; International Patent Publication No. WO 02/060937; International Patent Publication WO 2004/016148, and U.S. Pat. No. 6,613,882, all by the present Applicants. Each of these documents is incorporated by reference in its entirety).

In other embodiments, the targeting moiety is a functional equivalent of the Shiga toxin B-subunit. The term “functional equivalent”, as used herein, means any sequence derived from the B-subunit by mutation, deletion or addition, and with substantially the same routing properties as the B-subunit.

More precisely, a functional equivalent can be constituted by any fragment with the same retrograde transport properties and even intracellular transport to the nucleus as those described for the B-subunit. Examples include, but are not limited to, the B-subunit of verotoxin, described, for example, in S. B. Carderwood et al., Proc. Natl. Acad. Sci. USA, 1987, 84: 4365-4368 and International Patent Publication No. WO 1999/59627, and the B-subunit of ricin described, for example, in F. I. Lamb et al., Eur. J. Biochem., 1995, 148: 265-270. After describing the particular transport properties of such fragments, the skilled person will be able to select the fragment which would be the best candidate as a vector for routing any therapeutic moiety in any cellular compartment.

The capacity of polypeptidic sequence to bind specifically to the Gb3 receptor may be evaluated by the following assay which is based on the method described by Tarrago-Trani (Protein extraction and purification 2004, 39: 170-1760 and involves an affinity chromatography on a commercially available galabiose-linked agarose gel (Calbiochem). Galabiose (Gala1-4Gal) is the terminal carbohydrate portion of the oligosaccharide moiety of Gb3 and is thought to represent the minimal structure recognized by the B-subunit of Shiga toxin. The protein of interest in PBS buffer (500 μL) is mixed with 100 μL of immobilized galabiose resin previously equilibrated with the same buffer, and incubated for 30 minute to 1 hour at 4° C. on a rotating wheel. After a first centrifugation at 5000 rpm for 1 minute, the pellet is washed twice with PBS. The bound material is then eluated twice by re-suspending the final pellet in 2×500 μL of 100 mM glycine pH 2.5. Samples corresponding to the flow-through, the pooled washes and the pooled eluates are then analysed by SDS Page, Coomassie staining and Western blotting.

Thus, the present invention encompasses the use of the B-subunit of Shiga toxin or any other subunit or fragment of bacterial toxins which would have comparable activities, in particular routing properties analogous to those of the B-subunit, including polypeptides miming the Shiga toxin B-subunit. These polypeptides, and in general these functional equivalents, can be identified by screening methods which have in common the principle of detecting the interaction between random peptide sequences and the Gb3 receptor or soluble analogues of the receptor. By way of example, phage libraries expressing random peptide sequences for selection on affinity columns comprising Gb3 or after hybridization with soluble radioactive Gb3 analogues can be used.

Other examples of functional equivalents of Shiga toxin B-subunit include those polypeptides containing pre-determined mutations by, e.g., homologous recombination, site-directed or PCR mutagenesis, and the alleles or other naturally-occurring variants of the family of peptides and derivatives wherein the peptide has been covalently modified by substitution, chemical, enzymatic or other appropriate means with a moiety other than a naturally-occurring amino acid.

Shiga toxin B-subunit moieties and functional equivalents can be prepared using any of a wide variety of methods, including standard solid phase (or solution phase) peptide synthesis methods, as is known in the art. In addition, the nucleic acid encoding these peptides may be synthesized using commercially available oligonucleotide synthesis instrumentation and the proteins may be produced recombinantly using standard recombinant production systems.

As mentioned above, other suitable functional equivalents of the Shiga toxin B-subunit are peptide mimetics that mimic the three-dimensional structure of the naturally-occurring subunit. Such peptide mimetics may have significant advantages over naturally-occurring peptides including, for example, more economical production, greater chemical stability, enhanced pharmacological properties (half-life, absorption, potency, efficacy, etc), altered specificity (e.g., broad-spectrum biological activities, reduced antigenicity and others).

Generally, peptide mimetics are structurally similar to a paradigm polypeptide (i.e., a polypeptide that has a biochemical property or pharmacological activity), but have one or more peptide linkages optionally replaced by a non-peptide linkage. The use of peptide mimetics can be enhanced through the use of combinatorial chemistry to create drug libraries. The design of peptide mimetics can be aided by identifying amino acid mutations that increase or decrease the binding of a peptide to, for example, a tumor cell. Approaches that can be used include the yeast two hybrid method (see, for example, Chien et al., Proc. Natl. Acad. Sci. USA, 1991, 88: 9578-9582) and the phase display method.

In certain embodiments, it may be desirable to chemically modify the Shiga toxin B-subunit (or functional equivalent thereof) to facilitate coupling to the chemotherapeutic agent through the self-immolative spacer. Chemical modification may be performed to introduce one or more reactive functional groups on the Shiga toxin moiety. For example, the present Applicants have designed a Shiga toxin B-subunit (STxB) derivative, or mutant, called STxB-Cys. In this protein, a Cysteine was added at the C-terminus of mature STxB. The protein, when purified from bacteria, carries an internal disulfide bond, as wild type STxB After modification, STxB-Cys carries the free sulfhydryl group at the C-terminal Cys (International Publication No. WO 02/060937; M. Amessou et al., “Current Protocols in Cell Biology”, Eds. J. Bonifacino et al. (Eds.), Wiley: Hoboken, 2006, Chapter 15.10). Due to their nucleophilicity, free sulfhydryl groups are excellent acceptors for directed coupling approaches. Methods of introducing reactive functional groups on proteins and polypeptides are known in the art.

### C. Chemotherapeutics

In conjugates provided by the present invention, a Shiga toxin B-subunit moiety, or a functional equivalent thereof, is linked to a chemotherapeutics or anti-cancer agent, or a physiologically acceptable salt thereof. Suitable anti-cancer agents include any of a large variety of substances, molecules, compounds, agents or factors that are directly or indirectly toxic or detrimental to cancer cells.

As will be recognized by one of ordinary skill in the art, an anti-cancer agent suitable for use in the practice of the present invention may be a synthetic or natural compound; a single molecule or a complex of different molecules. Suitable anti-cancer agents can belong to any of a variety of classes of compounds including, but not limited to, small molecules, peptides, saccharides, steroids, antibodies (including fragments and variants thereof), fusion proteins, antisense polynucleotides, ribozymes, small interfering RNAs, peptidomimetics, and the like. Suitable chemotherapeutics for use in the present invention can also be found among any of a variety of classes of anti-cancer drugs including, but not limited to, alkylating agents, intercalating agents, topoisomerase I inhibitors, anti-metabolite drugs, anti-mitotic antibiotics, alkaloidal anti-tumor agents, hormones and anti-hormones, interferon, non-steroidal anti-inflammatory drugs, and various other anti-tumor agents such as kinase inhibitors, proteasome inhibitors, and NF-κB inhibitors.

Particularly suitable anti-cancer agents are agents that cause undesirable side effects due to poor selectivity/specificity for cancer cells; agents that undergo no or poor cellular uptake and/or retention; agents that are associated with cellular drug resistance; and agents that cannot be readily formulated for administration to cancer patients due to poor water solubility, aggregation, and the like.

Chemotherapeutic moieties suitable for use in the present invention may have any degree of cytotoxic activity when administered in an unconjugated form. For example, chemotherapeutic moieties may be selected among anti-cancer agents that have an activity comprised between about 1 nM and about 100 μM, e.g., between about 1 nM and about 100 nM, or between about 50 nM to about 500 nM, or between about 100 nM and about 1 μM, between about 500 nM and 2 μM, or between about 1 μM and about 10 μM, or between about 10 μM and 50 μM, or between about 25 μM and about 75 μM, or between about 50 μM and about 100 μM. In certain embodiments, the inventive STxB targeting approach can be efficiently used with chemotherapeutics that exhibit relatively low activity (in the upper nM range). This differentiates the STxB technology provided herein from targeting antibodies for which it is known in the field that they need to be used in combination with highly cytotoxic compounds.

Since conjugates of the present invention generally exhibit a high affinity for cancer cells that express the receptor Gb3, suitable chemotherapeutics may also be found among anti-cancer drugs that have been approved for cancers that are known to be associated with over-expression of Gb3. Thus, in certain embodiments, suitable anti-cancer agents are selected among drugs that are commonly used in the treatment of lymphomas, ovarian cancers, breast tumors, testicular cancers, colorectal cancers, intestine tumors, or astrocytomas.

An anti-cancer agent suitable for use in the practice of the present invention may, for example, be selected among taxanes, which are recognized as effective agents in the treatment of many solid tumors that are refractory to other anti-neoplastic agents. The two currently approved taxanes are placitaxel (TAXOL) and docetaxel (TAXOTERE). Paclitaxel, docetaxel, and other taxanes act by enhancing the polymerization of tubuline, an essential protein in the formation of spindle microtubules. This results in the formation of very stable, non-functional tubules, which inhibits cell replication and leads to cell death.

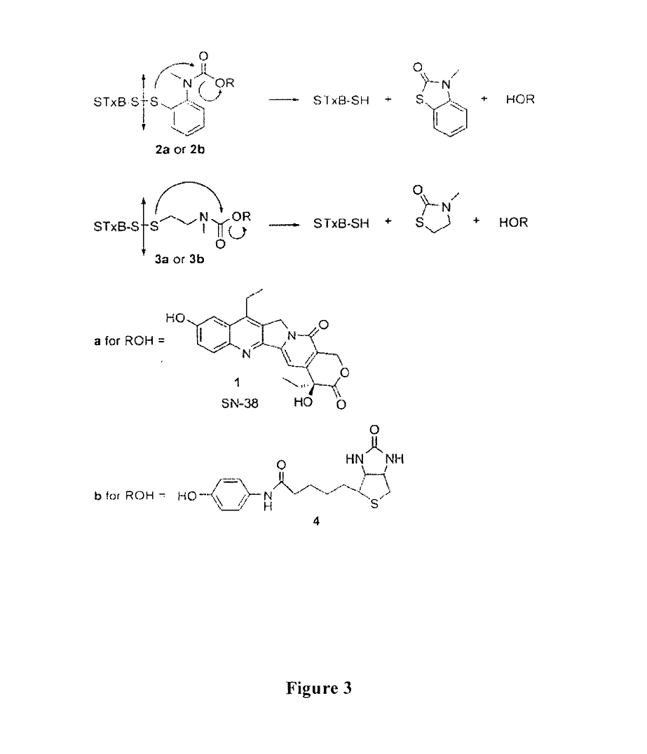

In another example, the chemotherapeutic moiety of an inventive conjugate is a topoisomerase inhibitor. Topoisomerase inhibitors are designed to interfere with the action of topoisomerase enzymes (topoisomerases I and II), which are enzymes that control the changes in DNA structure by catalyzing the breaking and rejoining of the phosphodiester backbone of DNA strands during the normal cell cycle. In recent years, topoisomerase inhibitors have become popular tools for cancer chemotherapy treatments. It is thought that topoisomerase inhibitors block the ligation step of the cell cycle, and that topoisomerase I and II inhibitors interfere with the transcription and replication of DNA by upsetting proper DNA supercoiling. Fluoroquinolones are a commonly prescribed class of topoisomerase inhibitors. Examples of topoisomerase I inhibitors include, but are not limited to, irinotecan and topotecan. Examples of topoisomerase II inhibitors include, but are not limited to, amsacrine, etoposide, etoposide phosphate, and tenipsode. The present Applicants have, for example, designed and developed STxB/SN-38 conjugates (see Examples 1-3). SN-38, the chemical structure of which is presented in FIG. 3, is the active principle of CPT11 (Campt), which is used in the treatment of colorectal carcinoma (E. Van Cutsem et al., Eur. J. Cancer, 1999, 35: 54). SN-38 belongs to the family of camptothecin derivatives that are cytotoxic by inhibition of topoisomerase I, and has been reported to be the most efficient compound in the family (B. Gatto et al., Curr. Pharm. Des., 1999, 5: 195). SN-38, which is poorly water soluble, cannot be used in vivo, hence the development of CPT11. In vitro, the cytotoxicity of STxB-SN38 was found to be much higher than the cytotoxicity of CPT11 (with IC₅₀ of 300 nm and 70 μM, respectively determined in HT-29 cells).

In another example, an anti-cancer agent suitable for use in the present invention is a ligand of the mitochondrial peripheral benzodiazepine receptor (mPBR). mPBR is involved in a functional structure designated as the permeability transition pore, which controls apoptosis. mPBR has been suggested as a putative target for therapeutic cell death induction. mPBR is overexpressed in some tumors, and this overexpression has a negative prognostic impact on breast cancer and colorectal cancer. Binding of mPBR with synthetic ligands such as PK11195 or RO5-4864 has been shown to facilitate apoptosis induction in human tumor cells by a variety of chemotherapeutic agents, including doxorubicin, daunorubicin, etoposide, 5-fluorouracil, paclitaxel, docetaxel, colchicin, arsenicals, lonidamine, and bortezomib. mPBR ligands that can be used as chemotherapeutics in the practice of the present invention include, but are not limited to, RO5-4864, PK11195, and diazepam. The present Applicants have, for example, designed and developed an STxB/RO5-4864conjugate (see Example 4), and demonstrated that delivery of RO5-4864 by STxB increased the cytotoxic potential of the drug (with IC₅₀ of 40 μM for the non-conjugated RO5-4864 and 0.3 μM for STxB/RO5-4864 determined in HT-29 cells).

In another example, an anti-cancer agent within an inventive conjugate may belong to the enediyne family of antibiotics. As a family, the enediyne antibiotics are the most potent anti-tumor agents discovered so far. Some members of this family are 1000 times more potent than adriamycin, one of the most effective clinically used anti-tumor antibiotics (Y. S; Zhen et al., J. Antibiot., 1989, 42: 1294-1298). Thus, chemotherapeutics suitable for use in the present invention may be found among derivatives of neocarzinostain, C1027, maduropeptin, kedarcidin, N1999A, calicheamicin, dynemicin, esperamicin, and shishijimicin.

Other examples of suitable anti-cancer agents include poorly water-soluble chemotherapeutics such as tamoxifen and BCNU. Tamoxifen has been used with varying degree of success to treat a variety of estrogen receptor positive carcinomas such as breast cancer, endometrial carcinoma, prostate carcinoma, ovarian carcinoma, renal carcinoma, melanoma, colorectal tumors, desmoid tumors, pancreatic carcinoma, and pituitary tumors. In addition to being limited by poor water solubility, chemotherapy using tamoxifen can cause side effects such as cellular drug resistance. BCNU (1,3-bis(2-chloroethyl)-1-nitrosourea) is well known for its anti-tumor properties and, since 1972, it has been charted by the National Cancer Institute for use against brain tumors, colon cancer, Hodgins disease, lung cancer and multiple myeloma. However, the efficient use of this anti-cancer drug is also compromised by its low solubility.

A wide variety of anti-cancer agents associated with drug resistance are also suitable for use in the present invention. A non-limiting example is methotrexate. Methotrexate, an analogue of folic acid widely used cancer drug, blocks important steps in the synthesis of tetrahydrofolic acid which itself is a critical source of compounds utilized in the synthesis of thymidylate, a building block that is specific and therefore especially critical for DNA synthesis. Methotrexate-induced drug resistance is linked to a deficiency in cellular uptake of that drug.

Other examples of suitable anti-cancer agents include purine and pyrimidine analogs that are associated with drug resistance due to inadequate intracellular inactivation of the drug through loss of enzymatic activity. An example of such a purine analog is 6-mercaptopurine (6-MP). A common cause of tumor cell resistance to 6-MP is the loss of the enzyme hypoxanthine guanine phosphoribosyl transferase (HGPRT) which activates 6-MP into its corresponding nucleotide, 6-mercaptophosphoribosylpurine (6-MPRP), the lethal form of the drug. The resistance could be overcome if 6-MPRP itself could be introduced into the cell. Although this compound is commercially available, it has not yet been used therapeutically in cancer treatment because it is not adequately transported into living cells. Association of 6-MPRP to a toxin moiety according to the present invention would dramatically increase its ability to cross the cell membrane. Thioguanine is another example of anti-cancer agent that is associated with drug resistance due to lack of the enzyme HGPRT. Examples of pyrimidine analogs that are associated with drug resistance due to inadequate intracellular inactivation include cytosine arabinoside and adenosine arabinoside which are activated by the enzyme deoxycytidine kinase (DOCK) to the lethal forms cytosine diphosphate and adenosine diphosphate, respectively. A toxin moiety can be coupled to the activated form of such pyrimidine analogs according to the present invention to enhance their cellular uptake and overcome cellular drug resistance.

Anti-cancer agents suitable for use in the practice of the present invention may belong to the family of photosensitizers used in photodynamic therapy (PDT). In PDT, local or systemic administration of a photosensitizer to a patient is followed by irradiation with light that is absorbed by the photosensitizer in the tissue or organ to be treated. Light absorption by the photosensitizer generates reactive species (e.g., radicals) that are detrimental to cells. For maximal efficacy, a photosensitizer not only has to be in a form suitable for administration, but also in a form that can readily undergo cellular internalization at the target site, preferably with some degree of selectivity over normal tissues. While some photosensitizers (e.g., Photofrin®, QLT, Inc., Vancouver, BC, Canada) have been delivered successfully as part of a simple aqueous solution, such aqueous solutions may not be suitable for hydrophobic photosensitizer drugs, such as those that have a tetra- or polypyrrole-based structure. These drugs have an inherent tendency to aggregate by molecular stacking, which results in a significant reduction in the efficacy of the photosensitization processes. Approaches to minimize aggregation include liposomal formulations (e.g., for benzoporphyrin derivative monoacid A, BPDMA, Verteporfin®, QLT, Inc., Vancouver, Canada; and zinc phthalocyanine, CIBA-Geigy, Ltd., Basel, Switzerland), and conjugation of photosensitizers to biocompatible block copolymers (Peterson et al., Cancer Res., 1996, 56: 3980-3985) and/or antibodies (Omelyanenko et al., Int. J. Cancer, 1998, 75: 600-608).

Photosensitizers suitable for use in the present invention include, but are not limited to, porphyrins and porphyrin derivatives (e.g., chlorins, bacteriochlorins, isobacteriochlorins, phthalocyanines, and naphthalocyanines); metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid (R. W. Redmond and J. N. Gamlin, Photochem. Photobiol., 1999, 70: 391-475). Exemplary photosensitizers suitable for use in the present invention are described in U.S. Pat. Nos. 5,171,741; 5,171,749; 5,173,504; 5,308,608; 5,405,957; 5,512,675; 5,726,304; 5,831,088; 5,929,105; and 5,880,145 (each of which is incorporated herein by reference in its entirety).

Other examples of suitable chemotherapeutics include agents that are developed to modulate the expression of genes for the treatment of cancer in strategies known as “antisense”, “antigen”, and “RNA interference” (A. Kalota et al., Cancer Biol. Ther., 2004, 3: 4-12; Y. Nakata et al., Crit. Rev. Eukaryot. Gene Expr., 2005, 15: 163-182; V. Wacheck and U. Zangmeister-Wittke, Crit. Rev. Oncol. Hematol., 2006, 59: 65-73; A. Kolata et al., Handb. Exp. Pharmacol., 2006, 173: 173-196). These approaches utilize, for example, antisense nucleic acids, ribozymes, triplex agents, or short interfering RNAs (siRNAs) to block the transcription or translation of a specific mRNA or DNA of a target gene, either by masking that mRNA with an antisense nucleic acid or DNA with a triplex agent, by cleaving the nucleotide sequence with a ribozyme, or by destruction of the mRNA, through a complex mechanism involved in RNA-interference. In all of these strategies, mainly oligonucleotides are used as active agents, although small molecules and other structures have also been applied. While the oligonucleotide-based strategies for modulating gene expression have a huge potential for the treatment of some cancers, pharmacological applications of oligonucleotides have been hindered mainly by the ineffective delivery of these compounds to their sites of action within cancer cells. (P. Herdewijn et al., Antisense Nucleic Acids Drug Dev., 2000, 10: 297-310; Y. Shoji and H. Nakashima, Curr. Charm. Des., 2004, 10: 785-796; A. W Tong et al., Curr. Opin. Mol. Ther., 2005, 7: 114-124).

Thus, conjugates can be developed according to the present invention that comprise a Shiga toxin B-subunit and a nucleic acid molecule that is useful as anti-cancer agent. Suitable nucleic acid anti-cancer agents include those agents that target: genes associated with tumorgenesis and cell growth or cell transformation (e.g., proto-oncogenes, which code for proteins that stimulate cell division), angiogenic/antiangionic genes, tumor suppressor genes (which code for proteins that suppress cell division), genes encoding proteins associated with tumor growth and/or tumor migration, and suicide genes which induce apoptosis or other forms of cell death, especially suicide genes that are most active in rapidly dividing cells.

Examples of antisense oligonucleotides suitable for use in the present invention include, for example, those mentioned in the following reviews: R. A Stahel et al., Lung Cancer, 2003, 41: S81-S88; K. F. Pirollo et al., Pharmacol. Ther., 2003, 99: 55-77; A. C. Stephens and R. P. Rivers, Curr. Opin. Mol. Ther., 2003, 5: 118-122; N. M. Dean and C. F. Bennett, Oncogene, 2003, 22: 9087-9096; N. Schiavone et al., Curr. Pharm. Des., 2004, 10: 769-784; L. Vidal et al., Eur. J. Cancer, 2005, 41: 2812-2818; T. Aboul-Fadi, Curr. Med. Chem., 2005, 12: 2193-2214; M. E. Gleave and B. P. Monia, Nat. Rev. Cancer, 2005, 5: 468-479; Y. S. Cho-Chung, Curr. Pharm. Des., 2005, 11: 2811-2823; E. Rayburn et al., Lett. Drug Design & Discov., 2005, 2: 1-18; E. R. Rayburn et al., Expert Opin. Emerg. Drugs, 2006, 11: 337-352: I. Tamm and M. Wagner, Mol. Biotechnol., 2006, 33: 221-238 (each of which is incorporated herein by reference in its entirety). Other examples of suitable antisense oligonucleotides include, but are not limited to, olimerson sodium (also known as Genasense™ or G31239, developed by Genta, Inc., Berkeley Heights, N.J.), GEM-231 (HYB0165, Hybridon, Inc., Cambridge, Mass.), Affinitak (ISIS 3521 or aprinocarsen, ISIS pharmaceuticals, Inc., Carlsbad, Calif.), OGX-011 (Isis 112989, Isis Pharmaceuticals, Inc.), ISIS 5132 (Isis 112989, Isis Pharmaceuticals, Inc.), ISIS 2503 (Isis Pharmaceuticals, Inc.), GEM 640 (AEG 35156, Aegera Therapeutics Inc. and Hybridon, Inc.), ISIS 23722 (Isis Pharmaceuticals, Inc.), and MG98 and GTI-2040 (Lorus Therapeutics, Inc. Toronto, Canada).

Examples of interfering RNA molecules suitable for use in the present invention include, for example, the iRNAs cited in the following reviews: O. Milhavet et al., Pharmacol. Rev., 2003, 55: 629-648; F. Bi et al., Curr. Gene. Ther., 2003, 3: 411-417; P. Y. Lu et al., Curr. Opin. Mol. Ther., 2003, 5: 225-234; I. Friedrich et al., Semin. Cancer Biol., 2004, 14: 223-230; M. Izquierdo, Cancer Gene Ther., 2005, 12: 217-227; P. Y. Lu et al., Adv. Genet., 2005, 54: 117-142; G. R. Devi, Cancer Gene Ther., 2006, 13: 819-829; M. A. Behlke, Mol. Ther., 2006, 13: 644-670; and L. N. Putral et al., Drug News Perspect., 2006, 19: 317-324 (each of which is incorporated herein by reference in its entirety).

In certain embodiments, an inventive conjugate of the present invention comprises a nucleic acid anti-cancer agent that is a ribozyme. As used herein, the term “ribozyme” refers to a catalytic RNA molecule that can cleave other RNA molecules in a target-specific manner. Ribozymes can be used to downregulate the expression of any undesirable products of genes of interest. Examples of ribozymes that can be used in the practice of the present invention include, but are not limited to, Angiozyme™ (RPI.4610, Sima Therapeutics, Boulder, Colo.), a ribozyme targeting the conserved region of human, mouse, and rat vascular endothelial growth factor receptor (VGEFR)-1 mRNA, and Herzyme (Sima Therapeutics).

Other examples of suitable chemotherapeutics include, but are not limited to, Zyloprim, alemtuzmab, altretamine, amifostine, nastrozole, antibodies against prostate-specific membrane antigen (such as MLN-591, MLN591RL and MLN2704), arsenic trioxide, Avastin™ (bevacizumab), (or other anti-VEGF antibody), bexarotene, bleomycin, busulfan, carboplatin, celecoxib, chlorambucil, cisplatin, cisplatin-epinephrine gel, cladribine, cytarabine, daunorubicin, daunomycin, dexrazoxane, docetaxel, doxorubicin, Elliott's B Solution, epirubicin, estramustine, etoposide phosphate, etoposide, exemestane, fludarabine, 5-FU, fulvestrant, gemcitabine, gemtuzumab-ozogamicin, goserelin acetate, hydroxyurea, idarubicin, idarubicin, Idamycin, ifosfamide, imatinib mesylate, letrozole, leucovorin, leucovorin levamisole, melphalan, L-PAM, mesna, methotrexate, methoxsalen, mitomycin C, mitoxantrone, MLN518 or MLN608 (or other inhibitors of the fit-3 receptor tyrosine kinase, PDFG-R or c-kit), itoxantrone, paclitaxel, Pegademase, pentostatin, porfimer sodium, Rituximab (RITUXAN™), talc, tamoxifen, temozolamide, teniposide, VM-26, topotecan, toremifene, Trastuzumab (Herceptin™ or other anti-Her2 antibody), 2C4 (or other antibody which interferes with HER2-mediated signaling), tretinoin, ATRA, valrubicin, vinorelbine, or pamidronate, zoledronate or another bisphosphonates.

As will be recognized by one skilled in the art, the specific examples of anti-cancer drugs cited herein represent only a very small number of the anti-cancer agents that are suitable for use in the present invention. For a more comprehensive discussion of updated cancer therapies see, http://www.cancer.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

### D. Conjugates

As can be appreciated by one skilled in the art, a conjugate of the present invention can be designed to comprise any number of Shiga toxin B-subunit moieties and any number of chemotherapeutic moieties, associated to one another by different self-immolative spacers. The design of a conjugate will be influenced by its intended purpose(s) and the properties that are desirable in the particular context of its use. Selection of a self-immolative spacer and of a method of synthesis to attach a toxin moiety to a chemotherapeutic moiety through the spacer is within the knowledge of one skilled in the art and will depend on the nature of the toxin and chemotherapeutic moieties, the presence and nature of functional chemical groups on the different moieties involved, the desired stability of the resulting conjugate, and the like.

Administration of a chemotherapeutic conjugate of the present invention to a cancer patient may increase specificity of the chemotherapeutic for cancer cells, increase its cellular internalization by cancer cells, decrease cellular degradation of the chemotherapeutic by cancer cells, increase its accumulation at the target site, overcome drug resistance, increase its biological activity and/or prevent, limit or eliminate undesirable side effects and toxicity as compared with administration of the chemotherapeutic agent alone (i.e., in a unconjugated

### E. Labels

In certain embodiments, a conjugate according to the present invention may, optionally, be labeled. More specifically, the Shiga toxin B-subunit moiety and/or the chemotherapeutic moiety may be labeled (i.e., attached to a detectable moiety). The role of a label or detectable agent is to facilitate detection of the conjugate. Preferably, the detectable agent is selected such that it generates a signal which can be measured and whose intensity is related to the amount of conjugate.

Thus, in certain embodiments, the toxin moiety within an inventive conjugate is labeled. Labeling usually involves non-covalent attachment or covalent attachment (directly or indirectly through a spacer), of one or more labels, preferably to non-interfering positions on the peptide sequence. Such non-interfering positions are positions that do not participate in the specific binding of the toxin moiety to tumor cells and/or to the internalization of the toxin moiety to tumor cells. In preferred embodiments, labeling does not substantially interfere with the desired biological or pharmacological activity of the toxin moiety.

Any of a wide variety of detectable agents can be used in the practice of the present invention. Suitable detectable agents include, but are not limited to, various ligands; radionuclides; fluorescent dyes; chemiluminescent agents; microparticles; enzymes; colorimetric labels and the like. In certain embodiments, a toxin moiety is labeled with an isotope. For example a toxin moiety may be isotopically-labeled (i.e., may contain one or more atoms that have been replaced by an atom having an atomic mass or mass number different from the atomic mass or mass number usually found in nature) or an isotope may be attached to the toxin molecule. Examples of isotopes that can be incorporated into toxin moieties include isotopes of hydrogen, carbon, fluorine, phosphorous, iodine, copper, rhenium; indium, yttrium, technetium and lutetium (i.e., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu). In certain embodiments, the toxin moiety is labeled with a metal such as Gadolinium (Gd) either through a covalent bonding or through chelation.

Such labeled toxin moiety may be useful as radiotracers for position emission tomography (PET) imaging or for single photon emission computerized tomography (SPECT).

### II—Pharmaceutical Compositions and Formulations

Conjugates described herein may be administered per se or in the form of a pharmaceutical composition. Accordingly, the present invention provides pharmaceutical compositions comprising an effective amount of at least one inventive conjugate and at least one pharmaceutically acceptable carrier or excipient.

A conjugate, or a pharmaceutical composition thereof, may be administered in such amounts and for such a time as is necessary or sufficient to achieve at least one desired result. For example, an inventive conjugate or pharmaceutical composition thereof can be administered in such amounts and for such a time that it kills cancer cells, reduces tumor size, inhibits tumor growth or metastasis, treats various leukemias, and/or prolongs the survival time of mammals (including humans) with those diseases, or otherwise yields clinical benefit.

Pharmaceutical compositions, according to the present invention, may be administered using any amount and any route of administration effective for achieving the desired therapeutic effect.

The exact amount of pharmaceutical composition to be administered will vary from subject to subject, depending on the species, age, and general condition of the subject, the severity of the condition, and the like (see below).

The optimal pharmaceutical formulation can be varied depending upon the route of administration and desired dosage. Such formulations may influence the physical state, stability, rate of in vivo release, and rate of in vivo clearance of the administered compounds.

Pharmaceutical compositions of the present invention may be formulated in dosage unit form for ease of administration and uniformity of dosage. The expression “unit dosage form”, as used herein, refers to a physically discrete unit of conjugate (with or without one or more additional agents) for the patient to be treated. It will be understood, however, that the total daily usage of compositions of the present invention will be decided by the attending physician within the scope of sound medical judgment.

After formulation with one or more appropriate physiologically acceptable carrier(s) or excipient(s) in a desired dosage, pharmaceutical compositions of the present invention can be administered to humans or other mammals by any suitable route. Various delivery systems are known and can be used to administer such compositions, including, tablets, capsules, injectable solutions, etc. Methods of administration include, but are not limited to dermal, intradermal, intramuscular, intraperitoneal, intravenous, subcutaneous, intranasal, pulmonary, epidural, ocular, and oral routes. An inventive composition may be administered by any convenient or otherwise appropriate route, for example, by infusion or bolus injection, by absorption through epithelial or mucocutaneous linings (e.g., oral, mucosa, rectal and intestinal mucosa, etc.) and may be administered together with other biologically active agents. Administration can be systemic or local.

Injectable preparations, for example, sterile injectable aqueous or oleaginous suspensions may be formulated according to the known art using suitable dispersing or wetting agents, and suspending agents. A sterile injectable preparation may also be a sterile injectable solution, suspension or emulsion in a non-toxic parenterally acceptable diluent or solvent, for example, as a solution in 2,3-butanediol. Among the acceptable vehicles and solvents that may be employed are water, Ringer's solution, U.S.P. and isotonic sodium chloride solution. In addition, sterile, fixed oils are conventionally employed as a solution or suspending medium. For this purpose any bland fixed oil can be employed including synthetic mono- or di-glycerides. Fatty acids such as oleic acid may also be used in the preparation of injectable formulations. Sterile liquid carriers are useful in sterile liquid from compositions for parenteral administration.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Liquid pharmaceutical compositions which are sterile solutions or suspensions can be administered by, for example, intravenous, intramuscular, intraperitoneal or subcutaneous injection. Injection may be via single push or by gradual infusion (e.g., 30 minute intravenous infusion). Where necessary, the composition may include a local anesthetic to ease pain at the site of injection.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming micro-encapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions which are compatible with body tissues.

Liquid dosage forms for oral administration include, but are not limited to, pharmaceutically acceptable emulsions, microemulsions, solutions, suspensions, syrups, elixirs, and pressurized compositions. In addition to the active ingredient (i.e., the conjugate), the liquid dosage form may contain inert diluents commonly used in the art such as, for example, water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cotton seed, ground nut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, suspending agents, preservatives, sweetening, flavoring, and perfuming agents, thickening agents, colors, viscosity regulators, stabilizers or osmo-regulators. Suitable examples of liquid carriers for oral administration include water (partially containing additives as above; e.g., cellulose derivatives, such as sodium carboxymethyl cellulose solution), alcohols (including monohydric alcohols and polyhydric alcohols such as glycols) and their derivatives, and oils (e.g., fractionated coconut oil and arachis oil)).

Solid dosage forms for oral administration include, for example, capsules, tablets, pills, powders, and granules. In such solid dosage forms, the active ingredient is mixed with at least one inert, physiologically acceptable excipient or carrier such as sodium citrate or dicalcium phosphate and one or more of: (a) fillers or extenders such as starches, lactose, sucrose, glucose, mannitol, and silicic acid; (b) binders such as, for example, carboxymethylcellulose, alginates, gelatin, polyvinylpyrrolidone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents such as, for example, cetyl alcohol and glycerol monostearate; (h) absorbents such as kaolin and bentonite clay; and (i) lubricants such as talc, calcium stearate, magnesium stearate, solid polyethylene glycols, sodium lauryl sulfate, and mixtures thereof. Other excipients suitable for solid formulations include surface modifying agents such as non-ionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminium silicate, and triethanolamine. In the case of capsules, tablets and pills, the dosage form may also comprise buffering agents. The amount of solid carrier per solid dosage form will vary widely but preferably will be from about 25 mg to about 1 g.

Solid compositions of a similar type may also be employed as fillers in soft and hard-filled gelatine capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition such that they release the active ingredient(s) only, or preferentially, in a certain part of the intestinal tract, optionally, in a delayed manner. Examples of embedding compositions which can be used include polymeric substances and waxes.

In certain embodiments, it may be desirable to administer an inventive composition locally to an area of the body in need of treatment. This may be achieved, for example, and not by way of limitation, by local infusion during surgery, topically application, by injection, by means of a catheter, by means of suppository, or by means of a skin patch or stent or other implant.

For topical administration, a composition is preferably formulated as a gel, an ointment, a lotion, or a cream which can include carriers such as water, glycerol, alcohol, propylene glycol, fatty alcohols, triglycerides, fatty acid esters, or mineral oil. Other topical carriers include liquid petroleum, isopropyl palmitate, polyethylene glycol, ethanol (95%), polyoxyethylenemonolaurate (5%) in water, or sodium lauryl sulfate (5%) in water. Other materials such as antioxidants, humectants, viscosity stabilizers, and similar agents may be added as necessary. Percutaneous penetration enhancers such as Azone may also be included.

In addition, in certain instances, it is expected that inventive compositions may be disposed within transdermal devices place upon, in, or under the skin. Such devices include patches, implants, and injections which release the compound onto the skin, by either passive or active release mechanisms. Transdermal administrations include all administrations across the surface of the body and the inner linings of bodily passage including epithelial and mucosal tissues. Such administrations may be carried out using the present compositions in lotions, creams, foams, patches, suspensions, solutions, and suppositories (rectal and vaginal).

Creams and ointments may be viscous liquid or semisolid emulsions of either the oil-in-water or water-in-oil type. Pastes comprised of absorptive powders dispersed in petroleum or hydrophilic petroleum containing active ingredient(s) may also be suitable. A variety of occlusive devices may be used to release active ingredient(s) into the bloodstream such as a semi-permeable membrane covering a reservoir containing the active ingredient(s) with or without a carrier, or a matrix containing the active ingredient. Suppository formulations may be made from traditional materials, including cocoa butter, with or without the addition of waxes to alter the suppository's melting point, and glycerine. Water soluble suppository bases, such as polyethylene glycols of various molecular weights may also be used.

Materials and methods for producing various formulations are known in the art and may be adapted for practicing the subject invention.

### III—Dosages and Administration

A treatment according to the present invention may consist of a single dose or a plurality of doses over a period of time.

Administration may be one or multiple times daily, weekly (or at some other multiple day interval) or on an intermittent schedule. For example, an inventive pharmaceutical composition may be administered one or more times per day on a weekly basis for a period of weeks (e.g., 4-10 weeks). Alternatively, an inventive pharmaceutical composition may be administered daily for a period of days (e.g., 1-10 days) following by a period of days (e.g., 1-30 days) without administration, with that cycle repeated a given number of times (e.g., 2-10 cycles).

Administration may be carried out in any convenient manner such as by injection (subcutaneous, intravenous, intramuscular, intraperitoneal, or the like) or oral administration.

Depending on the route of administration, effective doses may be calculated according to the body weight, body surface area, or organ size of the subject to be treated. Optimization of the appropriate dosages can readily be made by one skilled in the art in light of pharmacokinetic data observed in human clinical trials. Final dosage regimen will be determined by the attending physician, considering various factors which modify the action of the drugs, e.g., the drug's specific activity, the severity of the damage and the responsiveness of the patient, the age, condition, body weight, sex and diet of the patient, the severity of any present infection, time of administration, the use (or not) of concomitant therapies, and other clinical factors. As studies are conducted using the inventive conjugates and pharmaceutical compositions, further information will emerge regarding the appropriate dosage levels and duration of treatment.

Typical dosages comprise 1.0 pg/kg body weight to 100 mg/kg body weight. For example, for systemic administration, dosages may be 100.0 ng/kg body weight to 10.0 mg/kg body weight. For direct administration to the site via microinfusion, dosages may be 1 ng/kg body weight to 1 mg/kg body weight.

It will be appreciated that pharmaceutical compositions of the present invention can be employed in combination with additional therapies (i.e., a treatment according to the present invention can be administered concurrently with, prior to, or subsequently to one or more desired therapeutics or medical procedures). The particular combination of therapies (therapeutics or procedures) to employ in such a combination regimen will take into account compatibility of the desired therapeutic and/or procedures and the desired therapeutic effect to be achieved.

For example, methods and compositions of the present invention can be employed together with other procedures including surgery, radiotherapy (e.g., γ-radiotherapy, electron beam radiotherapy, proton therapy, brachytherapy, and systemic radioactive isotopes), endocrine therapy, hyperthermia, and cryotherapy.

Alternatively or additionally, methods and compositions of the present invention can be employed together with other agents to attenuate any adverse effects (e.g., antiemetics, pain relievers, and anti-nausea drugs), and/or with other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.cancer.gov/, a list of the FDA approved oncology drugs at http://www.fda.gov/cder/cancer/druglistframe.htm, and The Merck Manual, Seventeenth Ed. 1999, the cancer therapeutics sections of which are hereby incorporated by reference.

Methods and compositions of the present invention can also be employed together with one or more further combinations of cytotoxic agents as part of a treatment regimen, wherein the combination of cytotoxic agents is selected from: CHOPP (cyclophosphamide, doxorubicin, vincristine, prednisone, and procarbazine); CHOP (cyclophosphamide, doxorubicin, vincristine, and prednisone); COP (cyclophosphamide, vincristine, and prednisone); CAP-BOP (cyclophosphamide, doxorubicin, procarbazine, bleomycin, vincristine, and prednisone); m-BACOD (methotrexate, bleomycin, doxorubicin, cyclophosphamide, vincristine, dexamethasone, and leucovorin); ProMACE-MOPP (prednisone, methotrexate, doxorubicin, cyclophosphamide, etoposide, leucovorin, mechloethamine, vincristine, prednisone, and procarbazine); ProMACE-CytaBOM (prednisone, methotrexate, doxorubicin, cyclophosphamide, etoposide, leucovorin, cytarabine, bleomycin, and vincristine); MACOP-B (methotrexate, doxorubicin, cyclophosphamide, vincristine, prednisone, bleomycin, and leucovorin); MOPP (mechloethamine, vincristine, prednisone, and procarbazine); ABVD (adriamycin/doxorubicin, bleomycin, vinblastine, and dacarbazine); MOPP (mechloethamine, vincristine, prednisone and procarbazine) alternating with ABV (adriamycin/doxorubicin, bleomycin, and vinblastine); MOPP (mechloethamine, vincristine, prednisone, and procarbazine) alternating with ABVD (adriamycin/doxorubicin, bleomycin, vinblastine, and dacarbazine); ChlVPP (chlorambucil, vinblastine, procarbazine, and prednisone); IMVP-16 (ifosfamide, methotrexate, and etoposide); MIME (methyl-gag, ifosfamide, methotrexate, and etoposide); DHAP (dexamethasone, high-dose cytaribine, and cisplatin); ESHAP (etoposide, methylpredisolone, high-dose cytarabine, and cisplatin); CEPP(B) (cyclophosphamide, etoposide, procarbazine, prednisone, and bleomycin); CAMP (lomustine, mitoxantrone, cytarabine, and prednisone); CVP-1 (cyclophosphamide, vincristine, and prednisone), ESHAP (etoposide, methylpredisolone, high-dose cytarabine, vincristine and cisplatin); EPOCH (etoposide, vincristine, and doxorubicin for 96 hours with bolus doses of cyclophosphamide and oral prednisone), ICE (ifosfamide, cyclophosphamide, and etoposide), CEPP(B) (cyclophosphamide, etoposide, procarbazine, prednisone, and bleomycin), CHOP-B (cyclophosphamide, doxorubicin, vincristine, prednisone, and bleomycin), CEPP-B (cyclophosphamide, etoposide, procarbazine, and bleomycin), and P/DOCE (epirubicin or doxorubicin, vincristine, cyclophosphamide, and prednisone).

Alternatively or additionally, methods of the present invention can be employed together with therapies involving administration of one or more bioactive agents selected from the group consisting of antibodies, growth factors (e.g., Tumor-Necrosis Factor (TNF), Colony Stimulating Factor (CSF), Granulocyte-Colony Stimulating Factor (G-CSF) or Granulocyte-Macrophage Colony Stimulating Factor (GM-CSF)), hormones (e.g., estrogens, androgens, progestins, and corticosteroids), cytokines, anti-hormones, xanthines, interleukins (e.g., IL-2), and interferons.

### IV—Indications

Compositions and methods of the present invention can be used to treat primary and/or metastatic cancers, and other cancerous conditions. For example, compositions and methods of the present invention should be useful for reducing size of solid tumors, inhibiting tumor growth or metastasis, treating various lymphatic cancers, and/or prolonging the survival time of mammals (including humans) suffering from these diseases.

Examples of cancers and cancer conditions that can be treated according to the present invention include, but are not limited to, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, haematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., oesophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant malonoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye and adnexa, thyroid, adrenal gland, and other endocrine glands and related structures, secondary and unspecified malignant neoplasms of lymph nodes, secondary malignant neoplasm of respiratory and digestive systems and secondary malignant neoplasms of other sites.

Cancers that can be advantageously treated using compositions and methods of the present invention are cancers that are associated with expression of the receptor Gb3. Gb3 has been shown to be expressed on a narrow range of committed B cells and associated B-cell lymphomas (J. Gordon et al., Blood, 1983, 62: 910-917; L. J. Murray et al., Int. J. Cancer, 1985, 36: 561-565; M. Mangeney et al., Eur. J. Immunol., 1991, 21: 1131-1140; E; Oosterwijk et al., Int. J; Cancer, 1991, 48: 848-854; A. Kalisiak et al., Int. J. Cancer, 1991, 49: 837-845; E. C. LaCasse et al., Blood, 94: 2901-2910). Ovarian hyperplasias (S. Arab et al., Oncol. Res., 1997, 9: 553-563), cell suspensions obtained from human breast tumors (E. C. LaCasse et al., Blood, 94: 2901-2910), testicular seminomas (C. Ohyama et al., Int. J. Cancer, 1990, 45: 1040-1044), colorectal carcinomas (O. Kovbasnjuk et al., Proc. Natl. Acad. Sci. USA, 2005, 102: 19087-19092), and other small intestine tumors of different origins (E. C. LaCasse et al., Blood, 94: 2901-2910) have been tested positive for Gb3. Gb3 was also observed to be markedly increased in cell lines derived from astrocytomas (S. Arab et al., Oncol. Res., 1999, 11: 33-39). Thus, in certain embodiments, compositions and methods of the present invention are used in the treatment of cancers of the group consisting of lymphomas, ovarian cancers, breast tumors, testicular cancers, colorectal cancers, intestine tumors, and astrocytomas.

In certain embodiments, compositions and methods of the present invention are used in the treatment of cancers of the colon and rectum (CRC). Cancers of the colon and rectum are the second leading cause of cancer-related mortality in North America, Europe and Australia. The aggressiveness of the disease is directly correlated with the ability of the primary tumor to invade distant organs, most frequently the liver, and the 5-year survival of patients with distant metastasis present at the time of diagnosis is less than 10%. Indeed, in colorectal cancer, liver metastases are present in 25% of cases at the time of initial diagnosis, and cause the death of the majority of all patients. Therefore, specific targeting of primary tumors and distant metastases for diagnostic and therapeutic purposes remains one of the principal challenges in oncology. It was recently reported that Gb3 expression in human colorectal cancer correlates with invasiveness and the ability to form metastases (O; Kovbasnjuk et al., Proc. Natl. Acad. Sci. USA, 2006, 102: 19087-19092).

The present Applicants have designed and prepared a conjugate comprising the tumor delivery tool STxB linked to the camptothecin derivative SN38, the active principle of CPT-11 (irinotecan), used in the clinical management of metastatic CRC (see Example 1). The Applicants have then demonstrated the anti-tumor effect of STxB-SN38 on cells in culture (see Example 2), and on two different mouse models: xenografted human tumors and spontaneous adenocarcinomas in Ras-APC mice (see Example 3).

Tumors that can be treated using compositions and methods of the present invention may be refractory to treatment with other chemotherapeutics. The term “refractory”, when used herein in reference to a tumor means that the tumor (and/or metastases thereof), upon treatment with at last one chemotherapeutics other than an inventive composition, shows no or only weak anti-proliferative response (i.e., no or only weak inhibition of tumor growth) after the treatment of such a chemotherapeutic agent—that is, a tumor that cannot be treated at all or only with unsatisfying results with other (preferably standard) chemotherapeutics). The present invention, where treatment of refractory tumors and the like is mentioned, is to be understood to encompass not only (i) tumors where one or more chemotherapeutics have already failed during treatment of a patient, but also (ii) tumors that can be shown to be refractory by other means, e.g., biopsy and culture in the presence of chemotherapeutics.

### V—Pharmaceutical Packs or Kits

In another aspect, the present invention provides a pharmaceutical pack or kit comprising one or more containers (e.g., vials, ampoules, test tubes, flasks or bottles) containing one or more ingredients of an inventive pharmaceutical composition, allowing administration of a conjugate of the present invention.

Different ingredients of a pharmaceutical pack or kit may be supplied in a solid (e.g., lyophilized) or liquid form. Each ingredient will generally be suitable as aliquoted in its respective container or provided in a concentrated form. Pharmaceutical packs or kits may include media for the reconstitution of lyophilized ingredients. Individual containers of the kit will preferably be maintained in close confinement for commercial sale.

In certain embodiments, a pharmaceutical pack or kit includes one or more additional approved therapeutic agent(s) (e.g., one or more anti-cancer agents, as described above). Optionally associated with such container(s) can be a notice or package insert in the form prescribed by a governmental agency regulating the manufacture, use or sale of pharmaceutical or biological products, which notice reflects approval by the agency of manufacture, use or sale for human administration. The notice of package insert may contain instructions for use of a pharmaceutical composition according to methods disclosed herein.

An identifier, e.g., a bar code, radio frequency, ID tags, etc., may be present in or on the kit. The identifier can be used for example, to uniquely identify the kit for purposes of quality control, inventory control, tracking movement between workstations, etc.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Some of the results reported below are described in a French Ph.D. thesis entitled “Synthèses et Evaluation Biologiques de Prodrogues du SN-38, du Paclitaxel et du RO5-4864 Utilisables dans le Cadre de Stratégies de Vectorization Par Enzyme Immunociblée ou par la Toxine de Shiga”, presented by Abdessamad El Alaoui on Dec. 18, 2006, and in a scientific paper: A. El Alaoui et al., Angew Chem. Int. Ed., 2007, 46: 6469-6472. The thesis and scientific paper are each incorporated herein in their entirety, including the Supplemental Information of the scientific paper.

### Example 1

### Preparation and Stability of STxB/SN-38 and STxB/Biotin Conjugates

Preparation. Two prodrugs were designed and prepared based on SN-38 (compound 1), the active principle of CPT11 (Campto), which is used in the treatment of colorectal carcinoma (E. Van Cutsem et al., Eur. J. Cancer, 1999, 35: 54). SN-38 belongs to the class of camptothecin derivatives, which are cytotoxic by inhibition of topoisomerase I, and is one of the most efficient compounds in this family (B. Gatto et al., Curr. Pharm. Des., 1999, 5: 195). For coupling SN-38 to the Shiga toxin moiety, an STxB variant with a thiol functionality, termed STxB-Cys, was used that was specifically designed for site-directed chemical cross-linking in the laboratory of the present Applicants (PCT Publication No. WO 02/060937; and M. Amessou et al., Current Protocols in Cell Biology, J. Bonifacino et al. (Eds.), Wiley, Hoboken, 2006, chap. 15.10).

The phenolic position of SN-38 was chosen to build self-immolative spacers that include disulfide bonds. After cleavage of these bonds and release of a free thiol function, the free phenol is released without any other external reactant. To this end, two different spacers that exhibit variable stabilities in biological systems were envisioned. One of the spacers comprises an aromatic ring; the other an aliphatic chain.

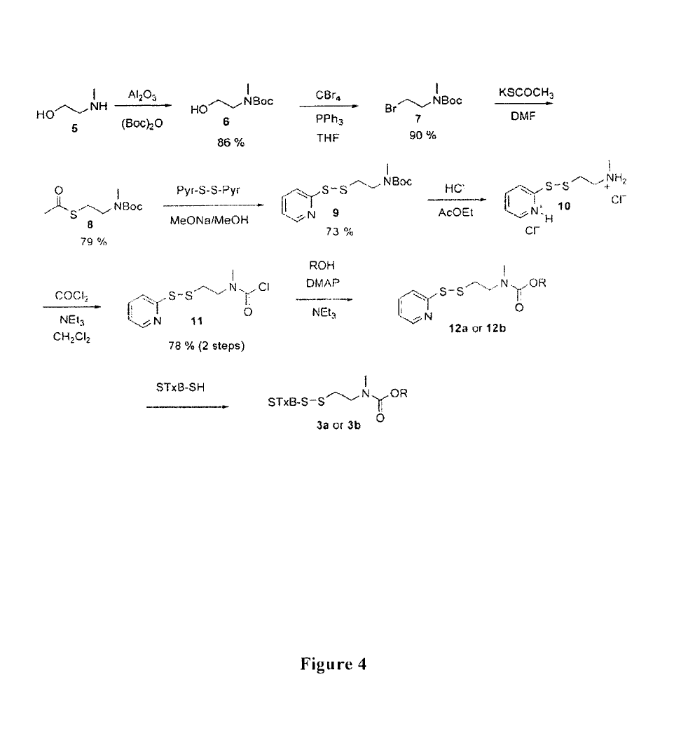

The two prodrugs (compounds 2 and 3) and the cleavage reactions they respectively undergo are depicted in FIG. 3. Two variants were synthesized for each spacer arm: one (a) with SN-38 (1), and the other (b) with biotin derivative 4. The latter compound allowed to circumvent the fact that release of SN-38 from compounds 2a and 3a could not be monitored in vivo because of lack of sensitivity. The biotin group was derivatized with a phenol spacer to obtain a similar susceptibility to cleavage as that with the phenol function of the SN-38. The compounds were obtained according to FIG. 4.

For the synthesis of compounds 3a and 3b, commercial amino alcohol 5 was first monoprotected as a tert-butoxycarbonyl (Boc) derivative. The hydroxyl group was converted to bromide with CBr₄ and then substituted by a thioacetate. The thiol function of 8 was activated as a pyridine disulfide 9. Liberation of the free amine was performed in acidic medium and the formed chlorhydrate 10 was kept as a salt because the free amine was unstable. Compound 10 was then reacted with phosgene and triethylamine to give the stable carbamoyl chloride 11. The phenol (SN-38 or biotin derivative) was first coupled in the presence of a stoichiometric amount of 4-dimethylaminopyridine (DMPA) to this bifunctional intermediate 11. Finally, STxB-Cys was reacted with carbamate 12 under basic conditions (pH 9).

The substitution levels of the coupling products were determined as 5 SN-38 or biotin molecules per STxB pentamer, using mass spectrometric analysis and fluorimetric dosage.

Stability. As a first step towards evaluation of the biological activity of compounds 2 and 3, the stability of the biotin versions was tested in different media. Compound 2b turned out to be readily activated even in the absence of cells, thus precluding its use in vivo. In contrast, compound 3b was completely stable over extended periods of up to 48 hours at 37° C. in all media, including pure fetal calf serum. Prodrug 3a was also stable in pure fetal calf serum, as shown by fluorimetric measurements.

### Example 2

### In vitro Activity of STxB/SN-38 Conjugate 3

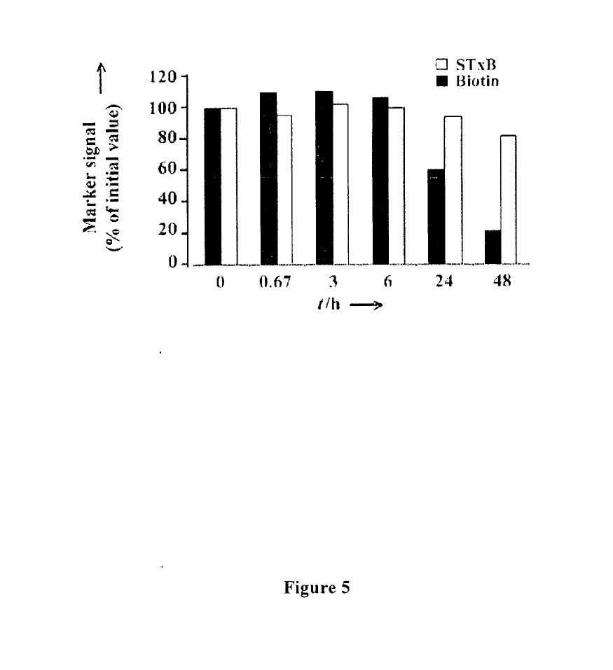

Compound 3 was chosen for an in-depth characterization on HT-29 colorectal carcinoma cells. ELISA analysis with 3b demonstrated that cleavage became detectable in the 6-24-h time interval, and was essentially complete at 48 hours (FIG. 5).

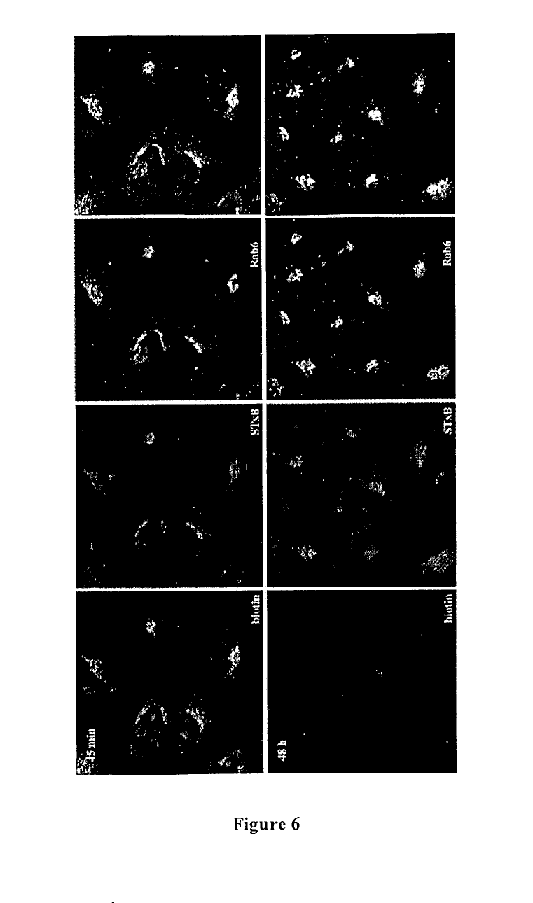

The same results were obtained using HeLa cells Immunofluorescence analysis was used to demonstrate that cleavage occurred intracellularly (FIG. 6). Consistent with ELISA data, no cleavage could be detected after short times of internalization (45 minutes), in which STxB (red) and biotin (green) co-localized with the Golgi marker Rab6 (blue). After 48 hours, STxB (red) could still be detected in the Golgi region (blue). However, the biotin signal was largely gone, which strongly suggests that reduction of the disulfide bond occurred with membranes of the biosynthetic/secretory pathway.

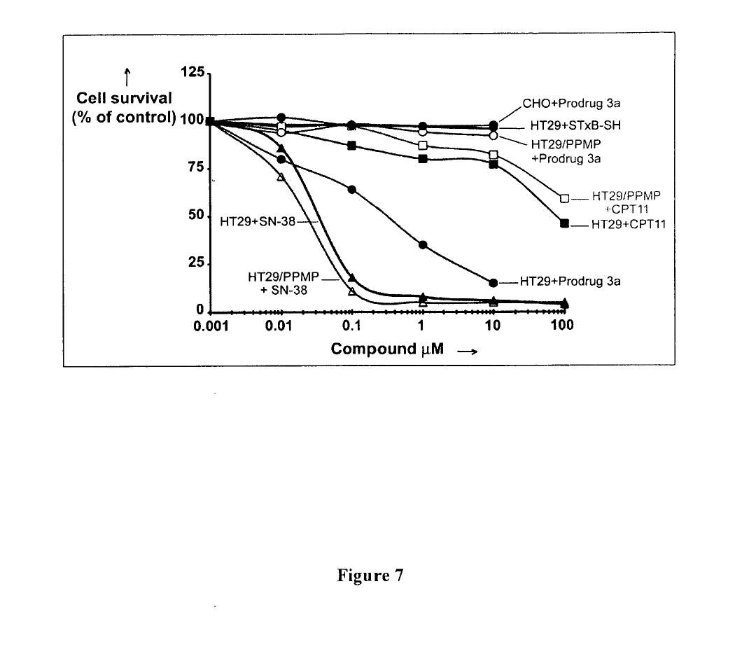

Having established that biotin model compound 3b is activated in HT-29 cells, the cytotoxic effect of corresponding prodrug 3a was determined As shown in FIG. 7, an IC₅₀ value of 300 nM (in SN-38 equivalents) was observed on Gb3-expressing HT-29 cells. To establish specificity, two Gb3 negative-control situations were tested: HT-29 cells that were treated with glycosylceramide synthase inhibitor 1-phenyl-2-palmitoylamino-3-morpholino-1-propanol (PPMP) (A. Abe et al., J; Biochem., 1992, 111: 191), or spontaneously Gb3-negative Chinese hamster ovary (CHO) cells. In both cases, the cells were not sensitive at all to incubation with prodrug 3a, which was used in the same concentration range for all experiments with 3a (FIG. 7). Furthermore, the non-derivatized STxB-SH was shown to have no measurable cytotoxicity on Gb3-expressing HT-29 cells under the experimental conditions used (FIG. 7).

Importantly, neither nonvectorized SN-38 (IC₅₀: 30 nM) nor its prodrug CPT-11 used in clinics (IC₅₀: 70 μM) had a cytotoxic effect on HT-29 cells that were dependent on Gb3 expression (FIG. 7), thus further establishing the selectivity of compound 3a for Gb3-expressing tumor cells.

In summary, the present Applicants have identified a novel tumor-delivery approach based on retrograde prodrug targeting to membranes of the biosynthetic/secretory pathway, by using STxB. The disulfide linkage of prodrug 3a is slowly released, most likely in the endoplasmic reticulum whose function in cellular redox homeostasis is well-recognized (A. Gorlach et al., Antioxid. Redox Signaling, 2006, 8: 1391). Retrograde delivery will place the site of drug release close to the nucleus, where the molecular target of hydrophobic SN-38 resides.

### Example 3

### In Vivo Activity of STxB/SN-38 Conjugate 3

Compound 3 was then investigated for its activity in vivo.

Protocol. Seventeen (17) APC^{1638N} mice of 6 months of age were injected 3 times intravenously at day (D)=1, 8, and 15 with 100 μg of STxB-SN38. As a control, mice were injected with STxB (n=6) at the same molar dose. At D=28 after the first injection, the mice were sacrificed, and their intestine was analyzed first macroscopically on autopsy preparations for the presence of periampular tumors. The same preparations were then also treated for pathological examinations.

Statistical Analysis. The presence of periampular tumors in STxB-SN38 treated and control mice was determined by macroscopical observation and pathological analysis. Table 1 presents experimental results obtained and expected results.

TABLE 1
Numbers of periampular tumors per total
number of mice that were analyzed.

All mice

| Conditions | Experimental results | Expected results’ |
|---|---|---|
| STxB-SN38 | 9/17 (53%) FT1, FT3* | 17/32 (53%)¹ FT1 |
| Control | 6/6 (93%) FT2, FT3 | 78/85 (92%)² FT2 |

Control mice were injected with STxB.
¹Expected results are deduced from in vivo tumor accumulation studies using fluorophore-labeled STxB. The underlying rational is that only tumors should be amenable to treatment by STxB-based therapy if they accumulate STxB in vivo. This is roughly only the case for 50% of them, due to heterogeneity in receptor expression (K. P. Jansen et al., Cancer Res., 2006, 66: 7230-7236).
²Expected results are deduced from historical analysis of the presence of periampular tumors in mice of the same genetic background.
*Fisher Test: FT1 = 1; FT2 = 1; FT3 = 0.05.

This experiment clearly established that STxB-SN38 injected mice were protected from tumor growth, within the limits by which STxB has access to these tumors, as established by in vivo bioaccumulation experiments (K. P. Jansen et al., Cancer Res., 2006, 66: 7230-7236). In contrast, periampular tumors developed in control mice, irrespective of the injection of STxB alone or CPT11, the clinical prodrug version of SN38.

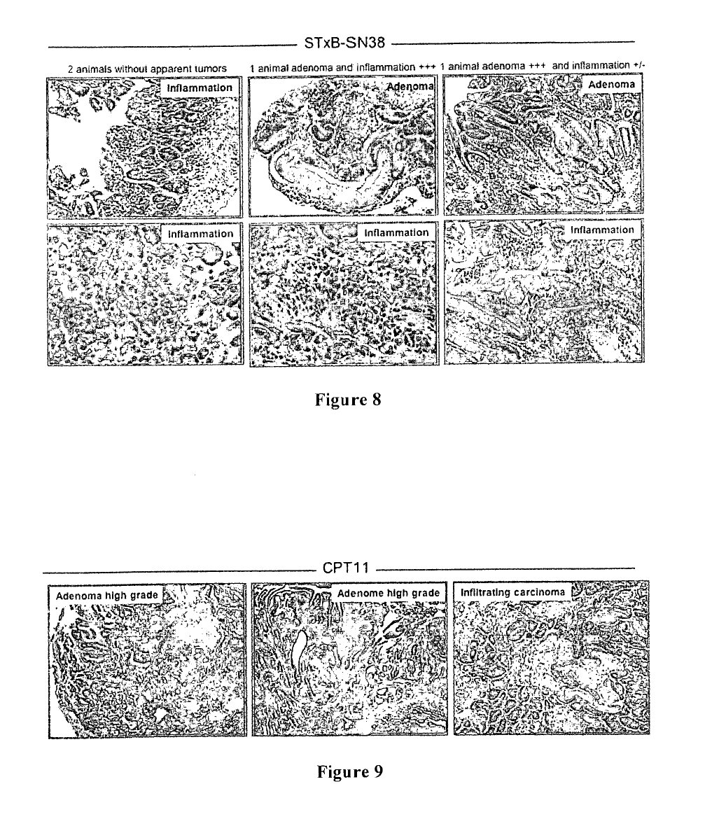

Pathology. Samples from STxB-SN38 injected mice (FIG. 8) or CPT11 injected control mice (FIG. 9) were prepared for H&E staining, and analyzed under the microscope. In STxB-SN38 injected mice, a strong inflammatory reaction was observed in the periampular region of mice in which no or only residual tumors could be detected. This strong inflammation could be interpreted as a “footprint” of the therapeutic response. In contrast, in CPT11 treated mice, high-grade adenomas and carcinomas could be detected. The inflammatory response was weak, except in the case of carcinoma.

### Example 4

### Preparation, Solubility and Cytotoxicity of STxB/RO5-4864 Conjugate

The present Applicants have also designed and developed STxB/RO5-4864: a conjugate constituted of a benzodiazepine precursor linked via a self-immolative spacer to the B-subunit of Shiga-toxin (the preparation and properties of which are described in a manuscript currently in preparation: A. El Alaoui et al., “Shiga toxin B-subunit solubilises and delivers benzodiazepine to tumor cells”).

RO5-4864 is a chloro derivative of diazepam, that is specific for the mitochondrial peripheral benzodiazepine receptor, mPBR, with Ki=23 nM. RO5-4864 exhibits very interesting in vitro and in vivo properties (Decaudin et al., Cancer Res., 2002, 62: 1388-1393). RO5-4864 itself is insoluble in aqueous solutions, but it is soluble in DMSO or ethanol. The Shiga toxin is freely soluble in water. The binding of both moieties according to the present invention yielded a conjugate with water solubility properties comparable to those of the free B-subunit of Shiga-Toxin. Cytotoxicity measurements were performed on HT-29 cells expressing Gb3 (Gb3+) and on HT-29 cells whose Gb3 expression was inhibited by the addition of PPMP (Gb3-) (Abe, J. Biochem., 1992, 111: 191). The IC₅₀ values were measured for the conjugate and compared to free RO5-4864. For free RO5-4864, the IC₅₀ value was found to be 40 μM, independently of Gb3 expression. For the conjugate, the value was 0.2 μM for Gb3+ cells and 10 μM for the Gb3-cells. The inventive conjugate was therefore found to be more cytotoxic than the free benzodiazepine, likely because of its increased solubility after conjugation to Shiga toxin B-subunit and due to cancer cell delivery.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A conjugate comprising at least one Shiga toxin B-subunit moiety, or a functional equivalent thereof, covalently attached to at least one chemotherapeutic moiety through a linker, wherein the linker comprises a self-immolative spacer.

2. The conjugate of claim 1, wherein the conjugate selectively interacts with cancer cells over normal cells.

3. The conjugate of claim 2, wherein the conjugate interacts with cancer cells that express Gb3.

4. The conjugate of claim 1, wherein the conjugate undergoes cellular internalization.

5. The conjugate of claim 4, wherein cellular internalization occurs via a retrograde pathway.

6. The conjugate of claim 1, wherein the chemotherapeutic moiety is selected from the group consisting of alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones, anti-androgens, ligands of mitochondrial peripheral benzodiazepine receptor, and any combinations thereof.

7. The conjugate of claim 1, wherein the chemotherapeutic moiety has a cytotoxic activity between 1 nM and 100 μM.

8. The conjugate of claim 1, wherein the self-immolative spacer comprises a disulfide bond.

9. The conjugate of claim 8, wherein the self-immolative spacer is an aliphatic self-immolative spacer comprising a disulfide bond.

10. The conjugate of claim 9, wherein the self-immolative spacer has the following formula: —S—S—(CH₂)₂—N(CH₃)—COO—.

11. The conjugate of claim 1, wherein the chemotherapeutic moiety comprises SN38.

12. The conjugate of claim 1, wherein the chemotherapeutic moiety comprises RO5-4864.

13. A pharmaceutical composition comprising an effective amount of at least one conjugate and at least one pharmaceutically acceptable carrier, wherein the conjugate comprises at least one Shiga toxin B-subunit moiety, or a functional equivalent thereof, covalently attached to at least one chemotherapeutic moiety through a linker, wherein the linker comprises a self-immolative spacer.

14. The pharmaceutical composition of claim 13 further comprising an additional therapeutic agent.

15. The pharmaceutical composition of claim 14, wherein the therapeutic agent is selected from the group consisting of an analgesic, an anesthetic, a haemostatic agent, an antimicrobial agent, an antibacterial agent, an antiviral agent, an antifungal agent, an antibiotic, an anti-inflammatory agent, an antioxidant, an antiseptic agent, an antihistamine agent, an antipruritic agent, an antipyretic agent, an immunostimulating agent, a dermatological agent, a anti-cancer agent, and any combination thereof.

16. The pharmaceutical composition of claim 13, wherein the composition is formulated to be administered intravenously or orally.

17. A method for treating a cancer or a cancerous condition in a subject in need thereof, the method comprising a step of:
administering to the subject an effective amount of a conjugate comprising at least one Shiga toxin B-subunit moiety, or a functional equivalent thereof, covalently attached to at least one chemotherapeutic moiety through a linker, wherein the linker comprises a self-immolative spacer.

18. The method of claim 17, wherein administration of the conjugate is carried out by topical, enteral or parenteral administration.

19. The method of claim 18, wherein administration of the conjugate is carried out by intravenous or oral administration.

20. The method of claim 17, wherein the cancer or cancerous condition is associated with overexpression of Gb3.

21. The method of claim 20, wherein the cancer or cancerous condition associated with overexpression of Gb3 is a member of the group consisting of lymphomas, ovarian cancers, breast tumors, testicular cancers, colorectal cancers, intestine tumors, and astrocytomas.

22. The method of claim 17 further comprising a step of:
administering to the subject an effective amount of an additional therapeutic agent.

23. The method of claim 22, wherein the therapeutic agent is an anti-cancer agent selected from the group consisting of an analgesic, an anesthetic, a haemostatic agent, an antimicrobial agent, an antibacterial agent, an antiviral agent, an antifungal agent, an antibiotic, an anti-inflammatory agent, an antioxidant, an antiseptic agent, an antihistamine agent, an antipruritic agent, an antipyretic agent, an immunostimulating agent, a dermatological agent, a anti-cancer agent, and any combination thereof.

24. A method for increasing selectivity of a chemotherapeutic agent for a cancer cell, the method comprising a step of:
covalently attaching the chemotherapeutic agent to a Shiga toxin B-subunit moiety, or a functional equivalent thereof, through a linker to form a conjugate, wherein the linker comprises a self-immolative spacer.

25. The method of claim 24, wherein the conjugate selectively interacts with cancer cells over normal cells.

26. The method of claim 25, wherein the conjugate interacts with cancer cells that express Gb3.

27. The method of claim 24, wherein the conjugate undergoes cellular internalization.

28. The method of claim 27, wherein cellular internalization occurs via a retrograde pathway.
